(19) United States
(12) Patent Application Publication
(10) Pub. No.: US 2012/0289706 A1
(43) Pub. Date: Nov. 15, 2012

(54) Method for Producing Triazolinthione Derivatives and Intermediates Thereof

(75) Inventors: Jens Renner, Dirmstein (DE); Jochen Dietz, Karlsruhe (DE); Thomas Grote, Wachenheim (DE); Joachim Gebhardt, Wachenheim (DE); Markus Nett, Schifferstadt (DE); Michael Keil, Freinsheim (DE)

(73) Assignee: BASF SE, Ludwigshafen (DE)

(21) Appl. No.: 13/515,064

(22) PCT Filed: Dec. 13, 2010
(86) PCT No.: PCT/EP10/69534

§ 371 Date: Jun. 11, 2012

(30) Foreign Application Priority Data
Dec. 18, 2009 (EP) 09179983.3

(51) Int. Cl. C07D 405/06; C07D 303/36

(52) U.S. Cl. 548/263.2; 549/552

(57) ABSTRACT

The present invention relates to a new process for the preparation of substituted thiotriazolo groups of the general formula (I)

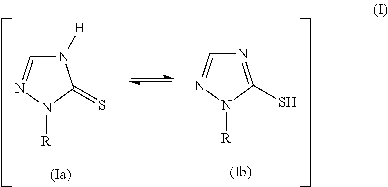

wherein the variables have the meanings as given in the claims and the description.

The present invention relates to a new general process for the preparation of thiotriazolo group-containing compounds. The invention furthermore relates to intermediates and to their preparation.

Important pesticidal compounds carry a thio-triazolo group. In the literature, several routes for the preparation of such thio-triazolo-compounds have been described. It is known, for example, to introduce the thio-group into the respective triazole compounds using a strong base such as LDA or n-BuLi and sulfur, preferably sulfur powder. Alternatively, the triazole compounds are reacted with sulfur in the presence of an aprotic polar solvent, such as, for example, an amide (such as dimethylformamide (DMF)) or N-alkylpyrrolidone (such as N-octylpyrrolidone, N-dodecylpyrrolidone or N-methylpyrrolidone (NMP)). See also WO 99/19307, WO 97/06151, WO 97/05119 and WO 96/41804. The disadvantages of these methods are that the yield of the desired products is usually comparably low and that the reaction conditions generally do not allow commercial scales.

WO 99/18088 (DE 19744400) is particularly directed to the synthesis of prothioconazole and analogues containing a thio-triazolo-group, wherein the respective hydrazine derivative or a salt thereof is, in a first step, reacted with a thiocyanate YSCN (Y=Na, K, NH₄⁺) in the presence of a diluting agent to the thiosemicarbazide derivative

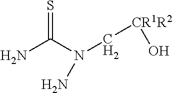

which is then, in a second step, reacted with formic acid in order to obtain the thiotriazolo-compound.

WO 99/18086 (DE 19744401) is directed to a further synthesis of prothioconazole and analogues containing a thio-triazolo-group, wherein in a first step the respective hydrazine derivative is reacted with a carbonyl compound R³C(═O)R⁴ and with thiocyanate XSCN(X=Na, K, NH₄⁺) to the triazolidinthion derivative,

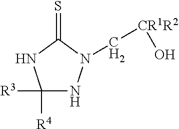

which is then in a second step reacted with formic acid in order to obtain the thiotriazolo-compound.

WO 99/18087 is directed to a further synthesis of prothioconazole and analogues containing a thio-triazolo-group, wherein in a first step the respective hydrazine derivative is reacted with formaldehyde and a thiocyanate XSCN in the presence of a diluting agent to the triazolidinthion derivative

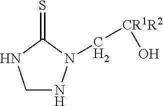

which is then in a second step either reacted with an oxidizing agent and a diluting agent or with formic acid.

WO 01/46158 is directed to a further synthesis of prothioconazole containing a thiotriazolo-group, wherein in a first step the respective hydrazine hydrochloride derivative is synthesized. In a second step the hydrazine hydrochloride derivative is first treated with alkali metal hydroxide in the presence of water and an aromatic hydrocarbon, then it is sequentially treated with formaldehyde and a thiocyanate XSCN(X=Na, K, NH₄⁺) in the presence of water and an aromatic hydrocarbon to result in a triazolidinthion derivative. In a third step said triazolidinthion derivative is oxidized using Fe(III)chloride in the presence of hydrochloric acid.

Pflanzenschutz-Nachrichten Bayer 57/2004, 2 gives a summary of different methods for the synthesis of prothioconazole.

Specific thio-triazole compounds that are known as active ingredients having pesticidal, in particular fungicidal activity, are known, for example, from WO 96/38440. Also WO 2009/077471 (PCT/EP2008/067483), WO 2009/077443 (PCT/EP2008/067394) WO 2009/077500 (PCT/EP2008/067545), WO 2009/077497 (PCT/EP2008/067539) describe further specific thio-triazolo compounds. Therein, several preparation routes for the disclosed compounds are explained.

The methods known from the literature are sometimes not suitable for the efficient synthesis of substituted thio-triazoles because the yield is not sufficient, the reaction has to be carried out in several steps and/or the reaction conditions and parameters such as temperature and/or reactants are not suitable for an upscale to industrially relevant amounts. For example the reaction that involves strong bases often result in a high amount of side products and low yields of the desired products. Inter alia because some thio-triazolo compounds are promising fungicidally active compounds, there is an ongoing need for improved processes that easily make the thio-triazolo compounds available.

It has now surprisingly been found a highly efficient general synthesis for building up substituted thio-triazolo group containing compounds. The inventive process represents a new general route for the cyclization of hydrazine derivatives in order to obtain thio-triazolo-compounds, wherein comparably high yields can be obtained. Furthermore, the inventive process can be carried out as a one-pot reaction and under conditions that are suitable for commercial scale-up.

The present invention relates to a process for the preparation of substituted thiotriazolo groups of the general formula (I)

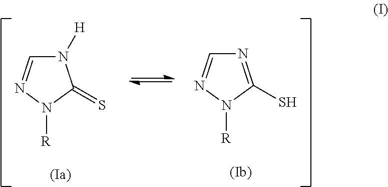

wherein a compound of formula (II)

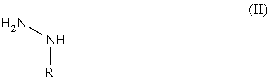

and/or a salt (IIa) thereof,

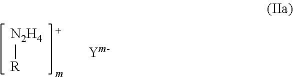

is reacted with Mⁿ⁺(SCN)ₙ (IV) and an orthoformic ester HC(OR)(OR⁵)(OR⁷) (V),
wherein the variables have the following meanings:
R is an organic group;
n 1, 2 or 3, depending on the meaning of M;
Mⁿ⁺ alkali metal cation (n=1), alkaline earth metal cation (n=2), Ag⁺ (n=1), Cu²⁺ (n=2), Co²⁺ (n=2), Cd²⁺ (n=2), Fe³⁺ (n=3); or [NR¹R²R³R⁴]⁺, wherein R¹, R², R³ and R⁴ independently from each other are selected from hydrogen and (C₁-C₁₀)-alkyl;
R⁵, R⁶, R⁷ independently from each other are selected from C₁-C₈-alkyl, C₂-C₈-alkenyl and C₂-C₈-alkinyl;
Y^{m-} is a counter anion of an organic acid or an inorganic acid and

m is 1, 2 or 3.

The thio-triazolo-groups of the general formula (I) can be present in two tautomeric forms—the “thiono” form of the formula (Ia) or in the “thiol” form of the formula (Ib).

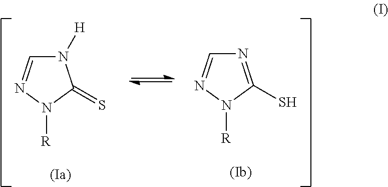

However, for the sake of simplicity, generally only one of the two forms, mostly the “thiol” form is shown here.

In some of the definitions of the symbols in the formulae given herein, collective terms are used which are generally representative of the following substituents:

halogen: fluorine, chlorine, bromine and iodine; alkyl and the alkyl moieties of composite groups such as, for example, alkylamino: saturated straight-chain or branched hydrocarbon radicals having 1 to 4, 6, 8 or 12 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methyl pentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: alkyl as mentioned above, where some or all of the hydrogen atoms in these groups are replaced by halogen atoms as mentioned above; in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl alkenyl and also the alkenyl moieties in composite groups, such as alkenyloxy: unsaturated straight-chain or branched hydrocarbon radicals having 2 to 4, 2 to 6 or 2 to 8 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: alkenyl as defined above, where some or all of the hydrogen atoms in these groups are replaced by halogen atoms as described above under haloalkyl, in particular by fluorine, chlorine or bromine;

alkadienyl: unsaturated straight-chain or branched hydrocarbon radicals having 4 to 6 or 4 to 8 carbon atoms and two double bonds in any position;

alkynyl and the alkynyl moieties in composite groups: straight-chain or branched hydrocarbon groups having 2 to 4, 2 to 6 or 2 to 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

haloalkynyl: alkynyl as defined above, where some or all of the hydrogen atoms in these groups are replaced by halogen atoms as described above under haloalkyl, in particular by fluorine, chlorine or bromine;

cycloalkyl and also the cycloalkyl moieties in composite groups: mono- or bicyclic saturated hydrocarbon groups having 3 to 8, in particular 3 to 6, carbon ring members, for example C₃-C₆-cycloalkyl, such as cyclopropyl, cyclobutyl, cyclopentyl, cyclohexyl;

halocycloalkyl: cycloalkyl as defined above, where some or all of the hydrogen atoms in these groups are replaced by halogen atoms as described above under haloalkyl, in particular by fluorine, chlorine or bromine;

cycloalkenyl: monocyclic monounsaturated hydrocarbon groups having preferably 3 to 8 or 4 to 6, in particular 5 to 6, carbon ring members, such as cyclopenten-1-yl, cyclopenten-3-yl, cyclohexen-1-yl, cyclohexen-3-yl, cyclohexen-4-yl and the like;

halocycloalkenyl: cycloalkenyl as defined above, where some or all of the hydrogen atoms in these groups are replaced by halogen atoms as described above under haloalkyl, in particular by fluorine, chlorine or bromine;

alkoxy: an alkyl group as defined above which is attached via an oxygen, preferably having 1 to 8, more preferably 2 to 6, carbon atoms. Examples are: methoxy, ethoxy, n-propoxy, 1-methylethoxy, butoxy, 1-methylpropoxy, 2-methylpropoxy or 1,1-dimethylethoxy, and also for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

haloalkoxy: alkoxy as defined above, where some or all of the hydrogen atoms in these groups are replaced by halogen atoms as described above under haloalkyl, in particular by fluorine, chlorine or bromine. Examples are OCH₂F, OCHF2, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

alkylene: divalent unbranched chains of CH₂ groups. Preference is given to (C₁-C₆)-alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄—CH₂;

a 3-, 4-, 5-, 6-, 7-, 8-, 9- or 10-membered saturated or partially unsaturated heterocycle which contains 1, 2, 3 or 4 heteroatoms from the group consisting of O, N and S, where the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. In particular:
a three- or four-membered saturated heterocycle (hereinbelow also referred to as heterocyclyl) which contains one or two heteroatoms from the group consisting of O, N and S as ring members;
a five- or six-membered saturated or partially unsaturated heterocycle which contains one, two, three or four heteroatoms from the group consisting of O, N and S as ring members: for example monocyclic saturated or partially unsaturated heterocycles which, in addition to carbon ring members, contain one, two or three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydro-oxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexa-hydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals;
a seven-membered saturated or partially unsaturated heterocycle which contains one, two, three or four heteroatoms from the group consisting of O, N and S as ring members: for example mono- and bicyclic heterocycles having 7 ring members which, in addition to carbon ring members, contain one, two or three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-,-2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1H]oxepin-2-, -3-, -4-, -5-, -6- or -7-yl, 2,3,4,7-tetrahydro[1H]oxepin-2-, -3- ,-4-, -5-, -6- or -7-yl, 2,3,6,7-tetrahydro[1H]oxepin-2-,
-3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding ylidene radicals;

a 5-, 6-, 7-, 8-, 9- or 10-membered aromatic heterocycle which contains 1, 2, 3 or 4 heteroatoms from the group consisting of O, N and S: in particular a five- or six-membered aromatic mono- or bicyclic heterocycle which contains one, two, three or four heteroatoms from the group consisting of O, N and S: the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. The heterocycle is in particular:
5-membered heteroaryl which contains one, two, three or four nitrogen atoms or one, two or three nitrogen atoms and/or one sulfur or oxygen atom, where the heteroaryl may be attached via carbon or nitrogen, if present: 5-membered heteroaryl groups which, in addition to carbon atoms, may contain one to four nitrogen atoms or one, two or three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
6-membered heteroaryl which contains one, two, three or four, preferably one, two or three, nitrogen atoms, where the heteroaryl may be attached via carbon or nitrogen, if present: 6-ring heteroaryl groups which, in addition to carbon atoms, may contain one to four or one, two or three nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The inventive process offers a general new and inventive route for the cyclization of hydrazine derivatives in order to obtain thio-triazolo-group-containing compounds. According to the present invention, R can be in principle any organic residue, as long as the hydrazine derivative can be reacted in the inventive manner. Thus “organic” residue” in the sense of the inventive process means, that the organic residue is inert under the conditions of the present invention. If necessary, some reactive groups can be protected via suitable protecting groups. It is within the skill of a person of the art to choose suitable groups and it is general knowledge of the skilled person how to insert and remove such groups. Important pesticidal compounds carry a thio-triazolo group. In particular, there are compounds of formula (I) known that are effective against phytopathogenic fungi. According to one aspect of the present invention, compounds of formula (I) are active compounds for controlling phytopathogenic fungi. Thus, compounds that can advantageously be synthesized using the new inventive process are for example fungicidal compounds of the azole compound class.

For example, the inventive process has shown to be very useful for the synthesis of thio triazole compounds that contain an epoxide group. Compounds that contain labile functional groups such as an epoxide group can often not be efficiently and/or economically be synthesized via prior art processes. Such compounds are for example described in WO 96/38440, WO 2009/077471 (PCT/EP2008/067483), WO 2009/077443 (PCT/EP2008/067394) WO 2009/077500 (PCT/EP2008/067545) and WO 2009/077497 (PCT/EP2008/067539), wherein these publications also describe the fungicidal activity of said compounds.

Accordingly, in one aspect of the inventive process, R in the compounds (I) (and the precursors thereof) has the following meaning (1):

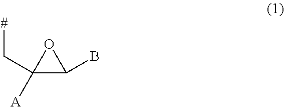

wherein # shall mean the point of attachment to the triazolo group or the hydrazine unit (or to the respective precursor-group), respectively, and A and B are as defined as follows:
A or B is a three-, four-, five-, six-, seven-, eight-, nine- or ten-membered saturated or partially unsaturated heterocycle or five-, six-, seven-, eight-, nine- or ten-membered aromatic heterocycle, where the heterocycle contains in each case one, two, three or four heteroatoms from the group consisting of O, N and S; is naphthyl or phenyl;

and the respective other variable B or A has
one of the meanings mentioned above for A or B or is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl or benzodioxolyl;
where A and/or B independently of one another are unsubstituted or substituted by one, two, three or four independently selected substituents L; wherein
   L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl-C₁-C₈-alkyl, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined below:
   n is 0, 1 or 2;
   A¹ is hydrogen, hydroxyl, C₁-C₈-alkyl, C₁-C₈-haloalkyl, amino, C₁-C₈-alkylamino or di-C₁-C₈-alkylamino,
   A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
   A³, A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
   where the aliphatic and/or alicyclic and/or aromatic groups of the radical definitions of L for their part may carry one, two, three or four identical or different groups R^{L}:
   R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino.

In the compounds (I), wherein R is a group (1) (also called compounds (I)-(1)) that can be advantageously be synthesized according to the invention, particular preference is given to the following meanings of the substituents, in each case on their own or in combination.

According to one embodiment, A and B independently stand for unsubstituted phenyl or substituted phenyl containing one, two, three or four independently selected substituents L.

According to one specific embodiment, A is unsubstituted phenyl.

According to a further embodiment, A is phenyl, containing one, two, three or four, in particular one or two, independently selected substituents L, wherein L is as defined or as preferably defined herein. According to one aspect of this embodiment, one of the substituents is in 4-position (para) of the phenyl ring. According to a further aspect, L is in each case independently selected from F, Cl, Br, nitro, phenyl, phenoxy, methyl, ethyl, iso-propyl, tert-butyl, methoxy, ethoxy, trifluoromethyl, trichloromethyl, difluoromethyl, difluorochloromethyl, trifluoromethoxy, difluoromethoxy and trifluorochloromethyl. According to another specific aspect, L is in each case independently selected from F, Cl and Br, in particular F and Cl.

According to another embodiment, A is monosubstituted phenyl, containing one substituent L, wherein L is as defined or as preferably defined herein. According to one aspect, said substituent is in para-position.

According to a specific embodiment, A is 3-fluorophenyl.

According to another embodiment, A is phenyl, containing two or three independently selected substituents L.

According to another preferred embodiment of the invention, A is phenyl which is substituted by one F and contains a further substituent L, where the phenyl may additionally contain one or two substituents L selected independently of one another, wherein L is as defined or preferably defined herein. According to a preferred embodiment, A is a group A-1

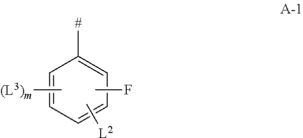

in which # is the point of attachment of the phenyl ring to the oxirane ring; and
L² is selected from the group consisting of F, Cl, NO₂, phenyl, halogenphenyl, phenoxy, halogenphenoxy, C₁-C₄-alkyl, C₁-C₄-haloalkyl, C₁-C₄-alkoxy, C₁-C₄-haloalkoxy and C₁-C₄-haloalkylthio;
L³ is independently selected from the group consisting of F, Cl, Br, NO₂, phenyl, halogenphenyl, phenoxy, halogenphenoxy, C₁-C₄-alkyl, C₁-C₄-alkoxy, C₁-C₄-haloalkoxy and C₁-C₄-haloalkylthio; and
m is 0, 1 or 2.

In one embodiment, L² is selected from the group consisting of F, Cl, methyl, methoxy, CF₃, CHF₂, OCF₃, OCF₃ and OCHF₂. According to a more specific embodiment, L² is F or Cl.

In one embodiment, L³ is independently selected from the group consisting of F, Cl, methyl, methoxy, CF₃, CHF₂, OCF₃, OCF₃ or OCHF₂. According to a more specific embodiment, L³ is independently F or Cl.

According to a preferred embodiment, m=0. According to a further preferred embodiment, m=1.

In the formula A-1, the fluorine substituent is, according to a preferred embodiment, in the 4-position.

According to still another embodiment, A is disubstituted phenyl, containing exactly two substituents L that are independently selected from each other, wherein L is as defined or as preferably defined herein. In particular, L is in each case independently selected from F, Cl, Br, C₁-C₄-alkyl, C₁-C₄-haloalkyl and C₁-C₄-alkoxy, in particular selected from F, Cl, C₁-C₄-alkyl, C₁-C₄-haloalkyl and C₁-C₄-alkoxy, in particular selected from F, Cl, methyl, trifluoromethyl and methoxy. According to a further aspect of this embodiment, the second substituent L is selected from methyl, methoxy and chloro. According to another aspect, one of the substituents is in the 4-position of the phenyl ring. According to another specific aspect, A is phenyl containing one F and exactly one further substituent L as defined or preferably defined herein.

According to yet a further preferred embodiment, A is disubstituted phenyl which contains one F and a further substituent L selected from the group consisting of Cl, C₁-C₄-alkyl, C₁-C₄-haloalkyl and C₁-C₄-alkoxy, in particular selected from the group consisting of Cl, methyl, trifluoromethyl and methoxy. The second substituent L is specifically selected from the group consisting of methyl, methoxy and chloroine. According to one aspect thereof, one of the substituents is located in the 4-position of the phenyl ring.

According to another specific embodiment, A is 2,4-disubstituted phenyl. According to still another specific embodiment, A is 2,3-disubstituted phenyl. According to still another specific embodiment, A is 2,5-disubstituted phenyl. According to still another specific embodiment, A is 2,6-disubstituted phenyl. According to still another specific embodiment, A is 3,4-disubstituted phenyl. According to still another specific embodiment, A is 3,5-disubstituted phenyl.

According to a further preferred embodiment of the invention, A is phenyl which is substituted by exactly two F. According to one aspect, A is 2,3-difluoro-substituted. According to a further aspect, A is 2,4-difluoro-substituted. According to yet a further aspect, A is 2,5-difluoro-substituted. According to yet a further aspect, A is 2,6-difluoro-substituted. According to yet a further aspect, A is 3,4-difluoro-substituted. According to yet a further aspect, A is 3,5-difluoro-substituted.

According to a further embodiment, A is trisubstituted phenyl containing exactly three independently selected substitutents L, wherein L is as defined or preferably defined herein. According to yet a further embodiment, A is phenyl which is substituted by exactly three F. According to one aspect, A is 2,3,4-trisubstituted, in particular 2,3,4-trifluoro-substituted. According to another aspect, A is 2,3,5-trisubstituted, in particular 2,3,5-trifluoro-substituted. According to still another aspect, A is 2,3,6-trisubstituted, in particular 2,3,6-trifluoro-substituted. According to still another aspect, A is 2,4,6-trisubstituted, in particular 2,4,6-trifluoro-substituted. According to still another aspect, A is 3,4,5-trisubstituted, in particular 3,4,5-trifluoro-substituted. According to still another aspect, A is 2,4,5-trisubstituted, in particular 2,4,5-trifluoro-substituted.

According to a preferred embodiment, B is phenyl, that is unsubstituted or phenyl which contains one, two, three or four independently selected substituents L, wherein L is as defined or preferably defined herein.

According to one embodiment of the invention, B is unsubstituted phenyl.

According to a further embodiment, B is phenyl which contains one, two, three or four independently selected substituents L, wherein L is as defined or preferably defined herein.

According to a further embodiment, B is phenyl which contains one, two or three, preferably one or two, independently selected substituents L, wherein L is as defined or preferably defined herein. According to a specific aspect, L is in each case independently selected from F, Cl, Br, methyl, methoxy and trifluoromethyl. According to still another embodiment, B is phenyl, which contains one, two or three, preferably, one or two, halogen substituents.

According to a further embodiment, B is phenyl which contains one, two, three or four substituents L, wherein L is independently selected from F, Cl, Br, methyl, ethyl, isopropyl, tert-butyl, methoxy, ethoxy, trifluoromethyl, trichloromethyl, difluoromethyl, difluorochloromethyl, trifluoromethoxy, difluoromethoxy and difluorochloromethyl. According to a specific aspect, L is in each case independently selected from F, Cl and Br.

According to still a further embodiment, B is unsubstituted phenyl or phenyl which contains one, two or three substituents independently selected from halogen, NO₂, amino, C₁-C₄-alkyl, C₁-C₄-alkoxy, C₁-C₄-haloalkyl, C₁-C₄-haloalkoxy, C₁-C₄-dialkylamino, thio and C₁-C₄-alkylthio.

According to a further embodiment, B is a phenyl ring that is monosubstituted by one substituent L, where according to a special aspect of this embodiment, L is located in the ortho-position to the point of attachment of the phenyl ring to the oxirane ring. L is as defined or preferably defined herein. According to a further specific embodiment, B is monochloro-substituted phenyl, in particular 2-chlorophenyl.

According to a further embodiment, B is phenyl, which contains two or three, in particular two, independently selected substitutents L, wherein L is as defined or preferably defined herein.

According to a further embodiment of the invention, B is a phenyl ring which contains a substituent L in the ortho-position and furthermore has one further independently selected substituent L. According to one aspect, the phenyl ring is 2,3-disubstituted. According to a further aspect, the phenyl ring is 2,4-disubstituted. According to yet a further aspect, the phenyl ring is 2,5-disubstituted. According to yet a further aspect, the phenyl ring is 2,6-disubstituted.

According to a further embodiment of the invention, B is a phenyl ring which contains a substituent L in the ortho-position and furthermore contains two further independently selected substituents L. According to one aspect, the phenyl ring is 2,3,5-trisubstituted. According to a further aspect, the phenyl ring is 2,3,4-trisubstituted. According to yet a further aspect, the phenyl ring is 2,4,5-trisubstituted.

In a further embodiment, B is phenyl which contains one substituent L in the 2-position and one, two or three further independently selected substituents L. According to a preferred embodiment, B is a group B-1

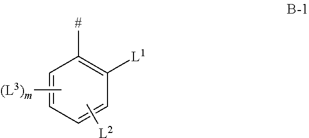

in which # denotes the point of attachment of the phenyl ring to the oxirane ring; and
L¹ is selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF₂, OCF₃, OCHF2 and SCF₃;
L² is selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF₂, OCF₃, OCHF₂ and SCF₃;
L³ is independently selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF2, OCF₃, OCHF₂ and SCF₃; and
m is 0, 1 or 2.

According to a preferred embodiment, L¹ is F. According to another preferred embodiment, L¹ is Cl. According to a further preferred embodiment, L′ is methyl. According to yet a further preferred embodiment, L¹ is methoxy. According to yet a further preferred embodiment, L¹ is CF₃. According to yet a further preferred embodiment, L¹ is OCF₃ or OCHF₂. According to a preferred embodiment, in the compounds of the formula I according to the invention, B is thus phenyl which contains a substituent selected from the group consisting of F, Cl, CH₃, OCH₃, CF₃, CHF2, OCF₃ and OCHF2 in the 2-position and one or two further independently selected substituents L.

According to a further preferred embodiment, L² is F. According to another preferred embodiment L² is Cl. According to a further preferred embodiment, L² is methyl. According to yet a further preferred embodiment, L² is methoxy. According to yet a further preferred embodiment, L² is CF₃. According to yet a further preferred embodiment, L² is OCF₃ or OCHF₂.

According to a preferred embodiment, L³ is F. According to another preferred embodiment, L³ is Cl. According to a further preferred embodiment, L³ is methyl. According to yet a further preferred embodiment, L³ is methoxy. According to yet a further preferred embodiment, L³ is CF₃. According to yet a further preferred embodiment, L³ is OCF₃ or OCHF₂.

According to a preferred embodiment, m=0; i.e. B is a disubstituted phenyl ring. According to a preferred aspect, B is a 2,3-disubstituted phenyl ring. According to a further preferred aspect, the phenyl ring B is 2,4-disubstituted. According to yet a further preferred aspect, the phenyl ring B is 2,5-disubstituted. According to yet a further preferred aspect, the phenyl ring is 2,6-disubstituted.

According to a further preferred embodiment, m=1; i.e. B is a trisubstituted phenyl ring. According to a preferred aspect, the phenyl ring B is 2,3,5-trisubstituted. According to another preferred further aspect, the phenyl ring B is 2,3,4-trisubstituted. According to yet a further preferred embodiment, the phenyl ring B is 2,4,5-trisubstituted.

Unless indicated otherwise, in group (1) L independently has the following preferred meanings:

According to one embodiment, L is independently selected from the group consisting of halogen, cyano, nitro, cyanato (OCN), C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, S-A¹, C(═O)A², C(═S)A², NASA; where A¹, A², A³, A⁴ are as defined below:
A¹ is hydrogen, hydroxy, C₁-C₄-alkyl, C₁-C₄-haloalkyl;
A² is one of the groups mentioned under A₁ or C₁-C₄-alkoxy, C₁-C₄-haloalkoxy, C₃-C₆-cycloalkyl, C₃-C₆-halocycloalkyl, C₃-C₆-cycloalkoxy or C₃-C₆-halocycloalkoxy;
A³, A⁴ independently of one another are hydrogen, C₁-C₄-alkyl, C₁-C₄-haloalkyl;
where the aliphatic and/or alicyclic groups of the radical definitions of L for their part may carry one, two, three or four identical or different groups R^{L}:
R^{L} is halogen, cyano, nitro, C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, amino, C₁-C₈-alkylamino, di-C₁-C₃-alkylamino.

Furthermore preferably, L is independently selected from the group consisting of halogen, NO₂, amino, C₁-C₄-alkyl, C₁-C₄-haloalkyl, C₁-C₄-alkoxy, C₁-C₄-haloalkoxy, C₁-C₄-alkylamino, di-C₁-C₄-alkylamino, thio and C₁-C₄-alkylthio.

Furthermore preferably, L is independently selected from the group consisting of halogen, C₁-C₄-alkyl, C₁-C₄-haloalkyl, C₁-C₄-alkoxy, C₁-C₄-haloalkoxy and C₁-C₄-haloalkylthio, in particular halogen, C₁-C₄-alkyl and C₁-C₄-haloalkyl.

According to a further preferred embodiment, L is independently selected from the group consisting of F, Cl, Br, CH₃, C₂H₅, i-C₃H₇, t-C₄H₉, OCH₃, OC₂H₅, CF₃, CCl₃, CHF2, CClF₂, OCF₃, OCHF₂ and SCF₃, in particular selected from the group consisting of F, Cl, CH₃, C₂H₅, OCH₃, OC₂H₅, CF₃, CHF₂, OCF₃, OCHF₂ and SCF₃. According to one aspect, L is independently selected from the group consisting of F, Cl, CH₃, OCH₃, CF₃, OCF₃ and OCHF₂. It may be preferred for L to be independently F or Cl.

According to one preferred embodiment, A and B are as defined as follows:

A phenyl, which is unsubstituted or substituted by one, two or three substituents L that may be the same or different, independently selected from F, Cl, Br, nitro, phenyl, phenoxy, methyl, ethyl, tert-butyl, methoxy, ethoxy, trifluoromethyl, trichloromethyl, difluoromethyl, difluorochloromethyl, trifluoromethoxy, difluoromethoxy and trifluoromethylthio; and

B phenyl, that is substituted by one, two or three substituents L that may be the same or different, independently selected from F, Cl, Br, methyl, ethyl, iso-propyl, tert-butyl, methoxy, ethoxy, trifluoromethyl, trichloromethyl, difluoromethyl, difluorochloromethyl, trifluoromethoxy, difluoromethoxy and trifluoromethylthio.

Specific compounds I that can advantageously be synthesized according to the present invention are compounds (I)-(1), wherein A and B are defined as follows:

A is phenyl, 4-chlorophenyl, 2,4-chlorophenyl, 2-chlorophenyl, 2-fluorophenyl, 4-fluorophenyl, 4-methylphenyl, 3-bromo-4-fluorophenyl, 4-bromophenyl, 3,4-dichlorophenyl, 4-tert-butyl-phenyl, 3-chlorophenyl, 3,5-dichlorophenyl or 4-trifluoromethoxyphenyl and B is 2-chlorophenyl. One specific compound is the compound where A is 4-flourphenyl and B is 2-chlorophenyl.

A is 4-fluorophenyl and B is 2-difluoromethoxyphenyl.

A is phenyl, 4-chlorophenyl, 2,4-chlorophenyl, 2-chlorophenyl, 2-fluorophenyl, 4-methylphenyl, 4-fluorophenyl, 3-bromo-4-fluorophenyl, 4-bromophenyl, 3,4-dichlorophenyl, 4-tert-butyl-phenyl, 3-chlorophenyl, 3,5-dichlorophenyl or 4-trifluoromethoxyphenyl, and B is 2-fluorophenyl.

A is phenyl, 4-chlorophenyl, 2,4-chlorophenyl, 2-chlorophenyl, 2-fluorophenyl, 4-methylphenyl, 4-fluorophenyl, 3-bromo-4-fluorophenyl, 4-bromophenyl, 3,4-dichlorophenyl, 4-tert-butyl-phenyl, 3-chlorophenyl, 3,5-dichlorophenyl or 4-trifluoromethoxyphenyl, and B is 2-bromophenyl.

Further specific compounds I that can advantageously be synthesized according to the present invention are compounds (I)-(1), wherein A and B are defined as follows:

A is 2,4-difluorophenyl and B is 2-chlorophenyl.

A is 3,4-difluorophenyl and B is 2-chlorophenyl.

A is 2,4-difluorophenyl and B is 2-fluorophenyl.

A is 3,4-difluorophenyl and B is 2-fluorophenyl.

A is 2,4-difluorophenyl and B is 2-trifluoromethylphenyl.

A is 3,4-difluorophenyl and B is 2-trifluoromethylphenyl.

A is 3,4-difluorophenyl and B is 2-methylphenyl

Further specific compounds I that can advantageously be synthesized according to the present invention are compounds (I)-(1), wherein A and B are defined as follows:

A is phenyl and B is 2,4-dichlorophenyl.

A is phenyl and B is 2-fluoro-3-chlorophenyl.

A is phenyl and B is 2,3,4-trichlorophenyl.

A is 4-fluorophenyl and B is 2,4-dichlorophenyl.

A is 4-fluorophenyl and B is 2-fluoro-3-chlorophenyl.

A is 4-fluorophenyl and B is 2,3,4-trichlorophenyl.

A is 2-chlorophenyl and B is 2,4-dichlorophenyl.

A is 2-chlorophenyl and B is 2-fluoro-3-chlorophenyl.

A is 2-chlorophenyl and B is 2,3,4-trichlorophenyl.

The meanings described above of the variables A, B and L for compounds (I) apply, unless indicated otherwise, correspondingly to the precursors of, the compounds (I) and side product IA.

According to the inventive process, the pure enantiomers or a mixture of enantiomers (racemic or enantiomerically enriched) of compounds of formula (II) and/or (IIa) can be used. According to a preferred embodiment, the racemic mixture is used. Depending on the use of the respective compound of formula (II) and/or (IIa), it is possible to obtain compounds of formula (I) having a certain stereochemistry. For example, the following different stereoisomers of compounds (I)-(1) can be obtained using the inventive process:

compound (I)-(1)-al):
Formula (I)-(1), wherein A is 4-fluoro-phenyl and B is 2-chlorophenyl
2-[(2S,3S)-3-(2-Chloro-phenyl)-2-(4-fluoro-phenyl)-oxiranylmethyl]-2H-[1,2,4]triazole-3-thiol (“cis”)
   compound (I)-(1)-a2):
   Formula (I)-(1), wherein A is 4-fluoro-phenyl and B is 2-chlorophenyl
2-[(2R,3R)-3-(2-Chloro-phenyl)-2-(4-fluoro-phenyl)-oxiranylmethyl]-2H-[1,2,4]triazole-3-thiol (“cis”)
   compound (I)-(1)-a3):
   Formula (I)-(1), wherein A is 4-fluoro-phenyl and B is 2-chlorophenyl
2-[(2S,3R)-3-(2-Chloro-phenyl)-2-(4-fluoro-phenyl)-oxiranylmethyl]-2H-[1,2,4]triazole-3-thiol (“trans”)
   compound (I)-(1)-a4):
   Formula (I)-(1), wherein A is 4-fluoro-phenyl and B is 2-chlorophenyl
2-[(2R,3S)-3-(2-Chloro-phenyl)-2-(4-fluoro-phenyl)-oxiranylmethyl]-2H-[1,2,4]triazole-3-thiol (“trans”)
   compound (I)-(1)-b1):
   Formula (I)-(1), wherein A is 2,4-difluoro-phenyl and B is 2-chlorophenyl
2-[(2S,3S)-3-(2-Chloro-phenyl)-2-(2,4-difluoro-phenyl)-oxiranylmethyl]-2H-[1,2,4]triazole-3-thiol (“cis”)
   compound (I)-(1)-b2):
   Formula (I)-(1), wherein A is 2,4-difluoro-phenyl and B is 2-chlorophenyl
2-[(2R,3R)-3-(2-Chloro-phenyl)-2-(2,4-difluoro-phenyl)-oxiranylmethyl]-2H-[1,2,4]triazole-3-thiol (“cis”)
   compound (I)-(1)-b3):
   Formula (I)-(1), wherein A is 2,4-difluoro-phenyl and B is 2-chlorophenyl
2-[(2S,3R)-3-(2-Chloro-phenyl)-2-(2,4-difluoro-phenyl)-oxiranylmethyl]-2H-[1,2,4]triazole-3-thiol (“trans”)
   compound (I)-(1)-b4):
   Formula (I)-(1), wherein A is 2,4-difluoro-phenyl and B is 2-chlorophenyl
2-[(2R,3S)-3-(2-Chloro-phenyl)-2-(2,4-difluoro-phenyl)-oxiranylmethyl]-2H-[1,2,4]triazole-3-thiol (“trans”)

With respect to the fungicidal activity of the end products, it may be preferred, if the “trans” diastereomers are synthesized in the inventive process.

One undesired side product in the synthesis of compounds (I)-(1) that may occur in undesired amounts with prior art processes and that can be reduced or even avoided using the new inventive process is the cyclizised hydroxy compound IA:

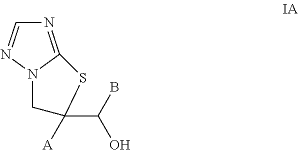

wherein A and B are as defined and preferably defined as for compounds (I)-(1). In conventional processes, for example using high temperature and/or strong bases, product IA may occur to up to 100%, leading, consequently, to very low yields of the desired product of formula I. According to the inventive process, side product IA is formed preferably to equal or less than 10%, more preferably equal or less than 8%, even more preferably equal or less than 5%.

Specifically, in the synthesis of compounds (I)-(1), wherein A and B are as defined in the following table A, the respective compounds IA-1 to IA-81 as individualized in table A, are undesired side products that are preferably reduced according to the inventive process.

TABLE A
specific compounds IA

| compound | A | B |
|---|---|---|
| IA-1 | 2-fluorophenyl | 2-chlorophenyl |
| IA-2 | 2-fluorophenyl | 3-chlorophenyl |
| IA-3 | 2-fluorophenyl | 4-chlorophenyl |
| IA-4 | 2-fluorophenyl | 2-methylphenyl |
| IA-5 | 2-fluorophenyl | 3-methylphenyl |
| IA-6 | 2-fluorophenyl | 4-methylphenyl |
| IA-7 | 2-fluorophenyl | 2-trifluoromethylphenyl |
| IA-8 | 2-fluorophenyl | 3-trifluoromethylphenyl |
| IA-9 | 2-fluorophenyl | 4-trifluoromethylphenyl |
| IA-10 | 3-fluorophenyl | 2-chlorophenyl |
| IA-11 | 3-fluorophenyl | 3-chlorophenyl |
| IA-12 | 3-fluorophenyl | 4-chlorophenyl |
| IA-13 | 3-fluorophenyl | 2-methylphenyl |
| IA-14 | 3-fluorophenyl | 3-methylphenyl |
| IA-15 | 3-fluorophenyl | 4-methylphenyl |
| IA-16 | 3-fluorophenyl | 2-trifluoromethylphenyl |
| IA-17 | 3-fluorophenyl | 3-trifluoromethylphenyl |
| IA-18 | 3-fluorophenyl | 4-trifluoromethylphenyl |
| IA-19 | 4-fluorophenyl | 2-chlorophenyl |
| IA-20 | 4-fluorophenyl | 3-chlorophenyl |
| IA-21 | 4-fluorophenyl | 4-chlorophenyl |
| IA-22 | 4-fluorophenyl | 2-methylphenyl |
| IA-23 | 4-fluorophenyl | 3-methylphenyl |
| IA-24 | 4-fluorophenyl | 4-methylphenyl |
| IA-25 | 4-fluorophenyl | 2-trifluoromethylphenyl |
| IA-26 | 4-fluorophenyl | 3-trifluoromethylphenyl |
| IA-27 | 4-fluorophenyl | 4-trifluoromethylphenyl |
| IA-28 | 2,3-difluorophenyl | 2-chlorophenyl |
| IA-29 | 2,3-difluorophenyl | 3-chlorophenyl |
| IA-30 | 2,3-difluorophenyl | 4-chlorophenyl |
| IA-31 | 2,3-difluorophenyl | 2-methylphenyl |
| 1A-32 | 2,3-difluorophenyl | 3-methylphenyl |
| IA-33 | 2,3-difluorophenyl | 4-methylphenyl |
| IA-34 | 2,3-difluorophenyl | 2-trifluoromethylphenyl |
| IA-35 | 2,3-difluorophenyl | 3-trifluoromethylphenyl |
| IA-36 | 2,3-difluorophenyl | 4-trifluoromethylphenyl |
| IA-37 | 2,4-difluorophenyl | 2-chlorophenyl |
| IA-38 | 2,4-difluorophenyl | 3-chlorophenyl |
| IA-39 | 2,4-difluorophenyl | 4-chlorophenyl |
| IA-40 | 2,4-difluorophenyl | 2-methylphenyl |
| IA-41 | 2,4-difluorophenyl | 3-methylphenyl |
| IA-42 | 2,4-difluorophenyl | 4-methylphenyl |
| IA-43 | 2,4-difluorophenyl | 2-trifluoromethylphenyl |
| IA-44 | 2,4-difluorophenyl | 3-trifluoromethylphenyl |
| IA-45 | 2,4-difluorophenyl | 4-trifluoromethylphenyl |
| IA-46 | 2,5-difluorophenyl | 2-chlorophenyl |
| IA-47 | 2,5-difluorophenyl | 3-chlorophenyl |
| IA-48 | 2,5-difluorophenyl | 4-chlorophenyl |
| IA-49 | 2,5-difluorophenyl | 2-methylphenyl |
| IA-50 | 2,5-difluorophenyl | 3-methylphenyl |
| IA-51 | 2,5-difluorophenyl | 4-methylphenyl |
| IA-52 | 2,5-difluorophenyl | 2-trifluoromethylphenyl |
| IA-53 | 2,5-difluorophenyl | 3-trifluoromethylphenyl |
| IA-54 | 2,5-difluorophenyl | 4-trifluoromethylphenyl |
| IA-55 | 2,6-difluorophenyl | 2-chlorophenyl |
| IA-56 | 2,6-difluorophenyl | 3-chlorophenyl |
| IA-57 | 2,6-difluorophenyl | 4-chlorophenyl |
| IA-58 | 2,6-difluorophenyl | 2-methylphenyl |
| IA-59 | 2,6-difluorophenyl | 3-methylphenyl |
| IA-60 | 2,6-difluorophenyl | 4-methylphenyl |
| IA-61 | 2,6-difluorophenyl | 2-trifluoromethylphenyl |
| IA-62 | 2,6-difluorophenyl | 3-trifluoromethylphenyl |
| IA-63 | 2,6-difluorophenyl | 4-trifluoromethylphenyl |
| IA-64 | 3,4-difluorophenyl | 2-chlorophenyl |
| IA-65 | 3,4-difluorophenyl | 3-chlorophenyl |
| IA-66 | 3,4-difluorophenyl | 4-chlorophenyl |
| IA-67 | 3,4-difluorophenyl | 2-methylphenyl |
| IA-68 | 3,4-difluorophenyl | 3-methylphenyl |
| IA-69 | 3,4-difluorophenyl | 4-methylphenyl |
| IA-70 | 3,4-difluorophenyl | 2-trifluoromethylphenyl |
| IA-71 | 3,4-difluorophenyl | 3-trifluoromethylphenyl |
| IA-72 | 3,4-difluorophenyl | 4-trifluoromethylphenyl |
| IA-73 | 3,5-difluorophenyl | 2-chlorophenyl |
| IA-74 | 3,5-difluorophenyl | 3-chlorophenyl |
| IA-75 | 3,5-difluorophenyl | 4-chlorophenyl |
| IA-76 | 3,5-difluorophenyl | 2-methylphenyl |
| IA-77 | 3,5-difluorophenyl | 3-methylphenyl |
| IA-78 | 3,5-difluorophenyl | 4-methylphenyl |
| IA-79 | 3,5-difluorophenyl | 2-trifluoromethylphenyl |
| IA-80 | 3,5-difluorophenyl | 3-trifluoromethylphenyl |
| IA-81 | 3,5-difluorophenyl | 4-trifluoromethylphenyl |

According to another embodiment of the present invention, R stands for a group of formula (2):

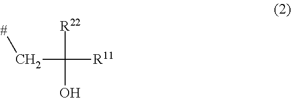

wherein R″ and R²² have the following meanings:
R¹¹, R²² independently of one another C₁-C₆-alkyl, C₁-C₆-haloalkyl, C₃-C₆-cycloalkyl, C₃-C₆-halocycloalkyl or phenyl, wherein the alkyl, cycloalkyl and phenyl moieties may be unsubstituted or substituted by one, two or three substituents L as defined or preferably defined above for compounds, wherein R is a group (1); or

R¹¹ and R²², together with the carbon atom to which they are attached, form a five- or six-membered saturated or partially unsaturated ring, that can be unsubstituted or substituted by one two three, four or five substituents L′, wherein L′ stands for L as defined above or stands for a group

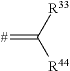

wherein R³³ and R⁴⁴ independently are selected from the group of hydrogen and the meaning for L as defined above.

According to one embodiment, R¹¹ and R¹² are preferably independently selected from C₁-C₄-alkyl and phenyl, wherein the alkyl and phenyl group independently may contain one, two, three or four substitutents, independently selected from F, Cl, Br, methoxy, ethoxy, propoxy, isopropoxy, C₁-C₂-alkoximino, cyclopropyl, cyclobutyl, cyclopentyl and/or cyclohexyl. Specifically, R¹¹ stands for C₁-C₄-alkyl that is substituted by one or two substituents independently selected from F, Cl, methoxy, cyclopropyl, cyclopentyl and/or cyclohexyl and R¹² stands for phenyl, that is substituted by one, two, three or four substituents independently selected from F, Cl, Br and methoxy. In one specific embodiment, R¹¹ is 1-ethyl that is 1-substituted by cyclopropyl and R¹² is 4-Chlorophenyl.

According to another embodiment, R¹¹ and R¹² are preferably independently selected from C₁-C₄-alkyl, phenyl-C₁-C₄-alkyl and C₃-C₃-cycloalkyl, preferably phenyl-C₁-C₄-alkyl and C₃-C₆-cycloalkyl, wherein the alkyl, phenyl and cycloalkyl groups independently may contain one, two, three or four substitutents, independently selected from F, Cl, Br, CN, methyl, ethyl, propyl, isopropyl and/or tert-butyl. Specifically, R¹¹ stands for phenyl-C₁-C₄-alkyl that is substituted in the phenyl moiety by one, two, three or four substituents independently selected from F, Cl and methoxy and R¹² stands for C₃-C₆-cycloalkyl, that is substituted by one, two, three or four substituents independently selected from F, Cl, Br and methoxy. In one specific embodiment, R¹¹ is 2-chlorophenylmethyl and R¹² is 1-chlorocyclopropyl.

According to still another embodiment, R¹¹ and R¹² are preferably independently selected from C₁-C₄-alkyl and phenyl-C₁-C₄-alkyl, wherein the alkyl and phenyl groups may contain one, two, three or four substitutents, independently selected from F, Cl, Br, CN, methyl, ethyl, propyl, isopropyl, tert-butyl, methoxy, ethoxy, methylthio, trifluoromethyl, trifluoromethoxy, trifluoromethylthio, chlorodifluoromethoxy, difluoromethoxy, chlorodifluoromethylthio, methoxycarbonyl, ethoxyvarbonyl, methoxyiminomethyl, 1-methoximinoethyl and nitro. Specifically, R¹¹ stands for C₁-C₄-alkyl that may be substituted by one or two substituents, independently selected from methyl, ethyl, propyl, isopropyl and tert-butyl and R¹² stands for phenyl-C₁-C₄-alkyl, that is substituted in the phenyl moiety by one, two, three or four substituents independently selected from F, Cl, Br, CN, methyl, trifluoromethyl and methoxy. In one specific embodiment R¹¹ is tert-butyl and R¹² is 2-(4-chlorophenyl)-1-ethyl.

According to still another embodiment, preferably R¹¹ and R²², together with the carbon atom to which they are attached, form a five- or six-membered saturated ring, that can be unsubstituted or substituted by one, two or three substituents L′. wherein L′ stands for L as defined above or stands for a group

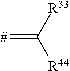

wherein R³³ and R⁴⁴ independently are selected from the group of hydrogen, C₁-C₄-alkyl and phenyl, wherein the alkyl and phenyl groups may contain one, two, three or four substitutents, independently selected from F, Cl, Br, CN, methyl, ethyl, propyl, isopropyl, tert-butyl, methoxy, ethoxy, methylthio, trifluoromethyl, trifluoromethoxy, trifluoromethylthio, chlorodifluoromethoxy, difluoromethoxy and nitro. Specifically, R¹¹ and R²², together with the carbon atom to which they are attached, form a five-membered saturated ring, that is substituted by one, two or three substituents L′, wherein L′ stands for C₁-C₄-alkyl or for a group

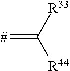

wherein R³³ and R⁴⁴ independently are selected from the group of hydrogen, C₁-C₄-alkyl and phenyl, wherein the alkyl and phenyl groups may contain one, two, three or four substitutents, independently selected from F, Cl, CN, methyl, isopropyl, tert-butyl and methoxy. In one specific embodiment R″ and R²², together with the carbon atom to which they are attached, form a five-membered saturated ring. that is substituted in 5-position by two methyl groups and contains a group

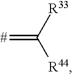

wherein R³³ is hydrogen and R⁴⁴ is 4-chlorophenyl in 2-position.

According to still another embodiment, R¹¹ and R²², together with the carbon atom to which they are attached, form a five- or six-membered saturated ring, that can be un-substituted or substituted by one, two or three substituents, independently selected from F, Cl, Br, CN, methyl, ethyl, propyl, isopropyl, tert-butyl, methoxy, ethoxy, methylthio, trifluoromethyl, trifluoromethoxy, trifluoromethylthio, chlorodifluoromethoxy, difluoromethoxy, nitro, benzyl, wherein the phenyl moiety itself may contain on, two, three or four substituents, independently selected from F, Cl, CN, methyl, isopropyl, tert-butyl and methoxy. In one specific embodiment R¹¹ and R²², together with the carbon atom to which they are attached, form a five-membered saturated ring, that is substituted in 5-position by two methyl groups and contains a 4-chlorobenzyl group in 2-position. Regarding compounds (I)-(2) and the synthesis of precursors thereof see also WO 96/16048 and WO 96/38423.

Consequently, according to a further embodiment, the inventive process is carried out using hydrazine compounds (II)-(2) and/or (IIa)-(2) as starting compounds:

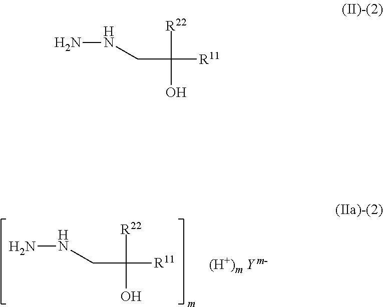

Compounds (II)-(2) can be obtained using a compound of formula (III)-(2) (R in formula (III) herein means a group (2), see above. Another way to obtain compounds (II)-(2) and (IIa)-(2) is to react an epoxide (VI)-(2) with hydrazine or hydrazine and the respective acid (H⁺)ₘY^{m-} such as HCl. Instead of hydrazine also hydrazine hydrate can be used (see above). Thereby, the epoxide ring is opened and the hydroxy group is formed:

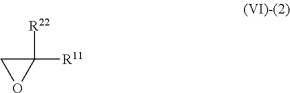

The reaction conditions that can be used are similar to those described in WO 00/146158.

According to one aspect of the present invention, the step for synthesizing the hydrazine derivative (II), wherein R stands for a group (2), from an epoxide (VI)-(2) is carried out before the inventive process for cyclization of said hydrazine derivative to the target thio-triazolo compound (I)-(2). According to another embodiment of the present invention, R stands for a group of formula (3):

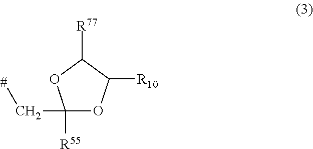

wherein R⁵⁵, R⁶⁶ and R⁷⁷ have the following meanings:

R⁵⁵ phenyl-C₁-C₈-alkyl, phenyl or a five- or six-membered saturated, partially unsaturated or aromatic heterocycle which contains one, two, three or four heteroatoms from the group consisting of O, N and S; where the aliphatic and/or aromatic and/or heterocyclic groups for their part may carry one, two, three or four identical or different groups selected from halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino, phenyl, halophenyl, phenyloxy, halophenyloxy;
R⁶⁶, R⁷⁷ independently of one another hydrogen, C₁-C₈-alkyl, C₁-C₈-haloalkyl, C₃-C₈-cycloalkyl, C₃-C₈-halocycloalkyl or phenyl, wherein the alkyl, cycloalkyl or phenyl moieties may be unsubstituted or substituted by one, two or three substituents selected from halogen, cyano, nitro, C₁-C₈-alkyl, C₁-C₈-haloalkyl, C₁-C₈-alkoxy, C₁-C₈-haloalkoxy

According to one embodiment, R⁵⁵ is phenyl, that is unsubstituted or substituted by one, two, three or four substituents independently selected from halogen, C₁-C₈-alkyl, C₁-C₈-haloalkyl, phenoxy-C₁-C₈-alkyl and halophenyloxy, and R⁶⁶ and R⁷⁷ are independently selected from hydrogen, methyl, ethyl, n-propyl and n-butyl. Specifically,

R⁵⁵ is phenyl, that contains one, two or three substituents independently selected from F, Cl and halophenoxy, wherein the phenoxy moiety contains one or two halogen atoms selected from Cl and F; and R⁶⁶ is hydrogen and R⁷⁷ is C₁-C₄-alkyl. In one specific embodiment R⁵⁵ is 4-(4-chlorophenoxy)-2-chlorophenyl, R⁶⁶ is hydrogen and R⁷⁷ is methyl.

Regarding compounds (I)-(3) and the synthesis of precursors thereof see also WO 96/41804.

According to another embodiment of the present invention, R stands for a group of formula (4):

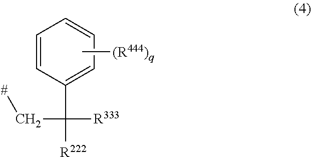

wherein R²²², R³³³ and R⁴⁴⁴ have the following meanings:

R²²² and R³³³ are independently selected from hydrogen, cyano, C₁-C₈-alkyl and C₁-C₆-haloalkyl, wherein the alkyl moieties may be unsubstituted or substituted by one, two, three or four substituents L as defined or preferably defined above for compounds, wherein R is a group (1). In particular, R²²² and R³³³ are independently selected from hydrogen, cyano and C₁-C₄-alkyl, wherein the alkyl moiety may contain one, two, three or four substituents independently selected from F, Cl, CN, C₁-C₄-alkoxy and C₁-C₄-haloalkoxy. q is one, two three or five, preferably one or two, and R⁴⁴⁴ are independently selected from L as defined or preferably defined above for compounds, wherein R is a group (1), in particular independently selected from F, Cl, CN, methyl, isopropyl, tert-butyl and methoxy, more specifically independently selected from Cl and F. According to one specific embodiment, R²²², is hydrogen, R³³³ is methyl, substituted by 1,1,2,2-tetrafluoroethoxy, and R⁴⁴⁴ is 2,4-dichlorophenyl. According to another specific embodiment, R²²² is cyano, R³³³ is n-butyl and R⁴⁴⁴ is 4-chlorophenyl. According to still another specific embodiment, R²²² is hydrogen, R³³³ is n-propyl and R⁴⁴⁴ is 2,4-dichlorophenyl. Regarding compounds (I)-(4) and the synthesis of precursors thereof see also DE19528300, DE19529089.

According to another embodiment of the present invention, R stands for a group of formula (5):

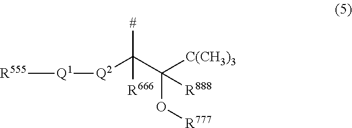

wherein # shall mean the point of attachment to the triazolo group and Q¹, Q², R⁵⁵⁵, R⁶⁶⁶, R⁷⁷⁷ and R⁵⁸⁸ are as defined as follows:

Q¹ O or a single bond to R⁵⁵⁵; in particular a single bond to R⁵⁵⁵;
Q² saturated hydrocarbon chain containing two to five carbon atoms, especially three carbon atoms, which may contain one, two or three substituents R^{z}, wherein R^{z} has the meaning:
   R^{z} halogen, C₁-C₈-alkyl, C₁-C₈-haloalkyl, C₂-C₈-alkenyl, C₂-C₈-haloalkenyl, C₁-C₈-alkoxy, C₁-C₈-haloalkoxy, C₁-C₈-alkylcarbonyloxy, C₂-C₈-alkenyloxy, C₂-C₈-haloalkenyloxy, C₃-C₈-cycloalkyl, C₃-C₈-halo cycloalkyl, C₃-C₈-cycloalkenyl, C₃-C₈-halocycloalkenyl, C₃-C₈-cycloalkoxy, C₃-C₆-cycloalkenyloxy, C₁-C₆-alkylen, oxy-C₂-C₄-alkylen, phenoxy, phenyl;

wherein R^{z} in each case is unsubstituted or contains one, two or three substituents, independently selected from L¹; R^{z} is in particular selected from halogen, C₁-C₄-alkyl, C₁-C₄-haloalkyl, C₁-C₄-alkoxy, C₁-C₄-haloalkoxy, C₃-C₈-cycloalkyl and C₃-C₆-halocycloalkyl;
R⁵⁵⁵ phenyl, which is unsubstituted or contains one, two, three, four or five independently selected substituents L¹, in particular one, two or three substituents L¹, wherein L¹ has in each case independently the meanings:
   L¹ halogen, C₁-C₈-alkyl, C₁-C₈-haloalkyl, C₂-C₈-alkenyl, C₂-C₈-haloalkenyl, C₁-C₈-alkadienyl, C₄-C₁₀-haloalkadienyl, C₁-C₈-alkoxy, C₁-C₈-haloalkoxy, C₁-C₈-alkylcarbonyloxy, C₂-C₈-alkenyloxy, C₂-C₃-haloalkenyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₈-cycloalkenyloxy, C₁-C₈-alkylen,
      the aliphatic and/or alicyclic and/or aromatic groups of the definitions of L¹ may contain one, two, three or four groups R^{L1} that are the same or different from each other:
      R^{L1} halogen, hydroxy, cyano, nitro, C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, C₃-C₈-cycloalkenyl, C₃-C₈-cycloalkoxy, C₃-C₈-halocycloalkoxy, C₁-C₆-alkylen, oxy-C₂-C₄-alkylen, oxy-C₁-C₃-alkylenoxy, C₁-C₈-alkylcarbonyl, C₁-C₈-alkylcarbonyloxy, C₁-C₈-alkoxycarbonyl, amino, C₁-C₁₀-alkylamino, di-C₁-C₈-alkylamino;
      in one preferred embodiment, L¹ is independently selected from F, Cl, C₁-C₄-alkyl, C₁-C₄-haloalkyl, C₁-C₄-alkoxy, C₁-C₄-haloalkoxy, C₃-C₆-cycloalkyl and C₃-C₆-halocycloalkyl;
R⁶⁶⁶ hydrogen, halogen, C₁-C₁₃-alkyl, C₁-C₁₀-haloalkyl, C₂-C₁₀-alkenyl, C₂-C₁₀-haloalkenyl, C₃-C₁₀-cycloalkyl, C₃-C₁₀-halocycloalkyl; in a specific embodiment, R⁶⁶⁶ is hydrogen
R⁷⁷⁷ hydrogen, C₁-C₁₀-alkyl, C₁-C₁₀-haloalkyl, C₂-C₁₀-alkenyl, C₂-C₁₀-haloalkenyl, C₂-C₁₀-alkinyl, C₃-C₁₀-cycloalkyl, C₃-C₁₀-halocycloalkyl, C₃-C₁₀-cycloalkenyl, C₃-C₁₀-halocycloalkenyl, tri-C₁-C₁₀-alkylsilyl; in a specific embodiment, R⁷⁷⁷ is hydrogen
R⁸⁸⁸ hydrogen, C₁-C₁₀-alkyl, C₂-C₁₀-alkenyl, C₂-C₁₀-halogenalkenyl, C₃-C₁₀-cycloalkyl; in a specific embodiment, R⁸⁸⁸ is hydrogen;
R⁶⁶⁶, R⁷⁷⁷ and R³³³ are, if it is not indicated otherwise, independently from each other unsubstituted or substituted with one, two, three, four or five L¹, as defined above.

For compounds (I)-(5) and their precursors (in particular wherein the triazole group does not contain SH or a derivatized sulfur group) and the preparation of the same, see WO2010/029001, WO2010/029002, WO 2010/029000, WO 2010/029003, WO2010/031721, WO 2010/031847, WO 2010/031848, PCT/EP 2009/062122 and/or PCT/EP2009/062909.

According to the inventive process, the thio-triazolo-cycle is built up starting from a substituted hydrazine (II) and/or a salt (IIa) thereof

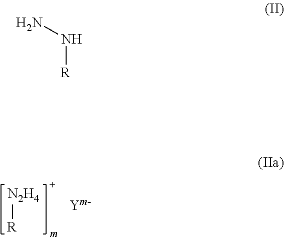

According to one embodiment of the inventive process, only the free hydrazine compound (II) is used as starting material. According to another embodiment of the inventive process, a salt (IIa) is used. According to still another embodiment of the invention, a mixture of the free hydrazine (II) and a salt (IIa) is used.

In formula (IIa), a proton (H⁺) is connected to a nitrogen atom of the hydrazine group, Y^{m-} is a counter anion of an organic acid or an inorganic acid and m is 1, 2 or 3. In the salt (IIa) the proton can be attached via either of the two nitrogen atoms. According to the inventive process, either one of the possible salt or a mixture of both can be used as starting compound in the inventive process:

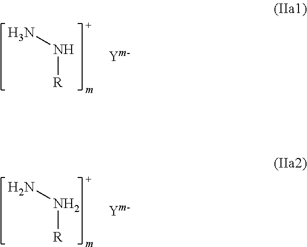

As suitable salts of the hydrazine derivative any salt formed with an inorganic or organic acid can be used as starting compound. Thus, Y^{m-} stands for the counter anion of the respective inorganic or organic acid, wherein “m” indicates the valency, i.e. 1, 2 or 3, depending on the kind of acid that is present as salt-forming acid. In principle, if the acid used has more than one proton, one or more protons can participate in forming the salt.

Examples of inorganic acids are hydrohalic acids, such as hydrogen fluoride, hydrogen chloride, hydrogen bromide and hydrogen iodide, carbonic acid, sulfuric acid, phosphoric acid, phosphonic acid, nitric acid, potassium hydrogen sulfate, sodium hydrogen sulfate, potassium dihydrogen phosphate, sodium dihydrogen phosphate, potassium hydrogen phosphate, sodium hydrogen phosphate. It may be preferred according to the present invention, to use hydrogen chloride, phosphoric acid, potassium dihydrogen phosphate, sodium dihydrogen phosphate, potassium hydrogen phosphate or sodium hydrogen phosphate.

Suitable organic acids are, for example, alkanoic acids, such as for example acetic acid, trifluoroacetic acid, trichloroacetic acid and propionic acid, and also glycolic acid, thiocyanic acid, lactic acid, succinic acid, citric acid, benzoic acid and other arylcarboxylic acids, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid etc. It may be preferred to use oxalic acid (H₂(CO₂)₂) in the inventive process.

The hydrazine derivative used in the inventive process can be synthesized from a compound of formula (III)

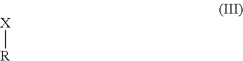

using hydrazine NH₂NH₂. Hydrazine may be used as free hydrazine (anhydrous), as hydrazine hydrate or hydrazine salt. Suitable hydrazine salts are for example monohydro chloric hydrazine or dihydrochloric hydrazine. When using hydrazine as a salt, it may be preferred to add a suitable base, which can be generally chosen by the skilled artisan, for example pyridine, triethylamine, potassium carbonate. X is a leaving group and R is as defined or preferably defined as for compounds (I) and/or (II). Possible leaving groups are for example halogen (e.g. Cl or Br) or OSO₂R′, wherein R′ is C₁-C₆-alkyl, C₁-C₆-haloalkyl, aryl or substituted aryl. In particular, OSO₂R′ stands for a mesylate-, triflate-, phenyl- or toluenesulfonate group.

The reaction conditions for preparing the hydrazine derivative are as generally used for such kind of substitution reactions. Possible diluents are for example all customary inert organic solvents. For examples alcohols such as methanol, ethanol or n-butanol can be used. Furthermore, ethers such as dioxane or methyl-tert-butylether can be used. Other suitable solvents are for example aromatic hydrocarbons such as benzene, toluene or xylol. Also any mixture of said solvents can be used. It is also possible to carry out the reaction without the addition of a separate diluting agent. In this case, hydrazine hydrate is used in excess being reactant and diluent at the same time.

According to one aspect of the present invention, the step for synthesizing the hydrazine derivative is carried out before the inventive process for cyclization of said hydrazine derivative to the target thio-triazolo group containing compound (I).

Temperatures that are suitable for synthesizing the hydrazine derivative can vary within a wide range. It may be preferred if the reaction mixture is held under reflux for a certain period of time. In general, temperatures of 40° C. to 130° C., in particular 60° C. to 90° C. are used.

In general, in the reaction for synthesizing the hydrazine derivative, for 1 mole of the reactant (III) 1 to 20 mole, in particular 5 to 10 mole, more specifically 2 to 8 moles of the hydrazine (or hydrazine hydrate or hydrazine salt) is used.

Generally, the reaction mixture comprising the desired hydrazine derivative can be worked up using well-known processes. In general, the obtained crude product can be used in the inventive process and there is no need to further purify the hydrazine derivative for this purpose.

As explained above, in the inventive process, also a salt (IIa) can be used as starting material. The salt can be prepared from a compound of formula (II) by reacting the same with the respective inorganic or organic acid. In case the chloride is to be synthesized, either aqueous HCl or HCl as a gas can be used. Any organic solvent that is suitable for this kind of reaction can be used as diluting agents. Examples are aromatic hydrocarbons such as benzene, toluene and xylol; ethers such as dioxane or methyl-tert-butylether. In general, the reaction can be carried out at ambient temperature, i.e. between 20 and 25° C. However, it may be advantageous in some cases to work at lower or higher temperatures.

In general, the hydrazine derivative (II) is dissolved in a suitable solvent and then the respective acid is added in an equimolar amount or in excess. The isolation of the product can be carried out according to processes well-known to the skilled person.

Some of the compounds of formula (IIa) are novel and are also an object of the present invention. In particular, the present invention also encompasses compounds of formula (IIa), wherein R stands for a group (1) as defined above (compounds (IIa)-(1)).

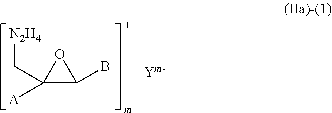

wherein A, B, Y and m are as defined as for compounds of formula (I) and (IIa), respectively. Thereby, also the preferred and specific embodiments for (1) (variables A and B) and (IIa) (variables Y and m) are analoguously preferred for compounds of formula (IIa)

The novel compounds according to the invention contain chiral centers and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers.

Accordingly, the invention provides both the pure enantiomers or diastereomers and mixtures thereof. This applies to the compounds according to the invention The scope of the present invention includes in particular the (R) and (S) isomers and the racemates of the compounds according to the invention, which have centers of chirality. Suitable compounds according to the invention also include all possible stereoisomers (cis/trans isomers) and mixtures thereof.

The compounds according to the invention may be present in various crystal modifications. They are likewise provided by the present invention.

In particular with a view to their use, preference is given to the compounds (IIa)-(1) according to the invention compiled in tables 1b to 137b below. The groups mentioned for a substituent in the tables are furthermore per se, independently of the combination in which they are mentioned, a particularly preferred aspect of the substituent in question.

### Table 1b

Compounds (IIa)-(1), wherein A is 2,3-difluorophenyl and the combination of B and Y^{m-} corresponds in each case to one row of table B (Compounds (IIa)-(1). 1bB-1 to (IIa)-(1).1bB-440)

### Table 2b

Compounds (IIa)-(1), wherein A is 2,4-difluorophenyl and the combination of B and Y^{m-} corresponds in each case to one row of table B (Compounds (IIa)-(1).2bB-1 to (IIa)-(1).2bB-440)

### Table 3b

Compounds (IIa)-(1), wherein A is 2,5-difluorophenyl and the combination of B and Y^{m-} corresponds in each case to one row of table B (Compounds (IIa)-(1).3bB-1 to (IIa)-(1).3bB-440)

### Table 4b

Compounds (IIa)-(1), wherein A is 2,6-difluorophenyl and the combination of B and Y^{m-} corresponds in each case to one row of table B (Compounds (IIa)-(1).4bB-1 to (IIa)-(1).4bB-440)

### Table 5b

Compounds (IIa)-(1), wherein A is 3,4-difluorophenyl and the combination of B and Y^{m-} corresponds in each case to one row of table B (Compounds (IIa)-(1).5bB-1 to (IIa)-(1).5bB-440)

### Table 6b

Compounds (IIa)-(1), wherein A is 3,5-difluorophenyl and the combination of B and Y^{m-} corresponds in each case to one row of table B (Compounds (IIa)-(1).6bB-1 to (IIa)-(1).6bB-440)

### Table 7b

Compounds (IIa)-(1), wherein A is 2-fluoro-3-chlorophenyl and the combination of B and Y^{m-} corresponds in each case to one row of table B (Compounds (IIa)-(1).7bB-1 to (IIa)-(1).7bB-440)

### Table 8b

Compounds (IIa)-(1), wherein A is 2-fluoro-4-chlorophenyl and the combination of B and Y^{m-} corresponds in each case to one row of table B (Compounds (IIa)-(1).8bB-1 to (IIa)-(1).8bB-440)

### Table 9b

Compounds (IIa)-(1), wherein A is 2-fluoro-5-chlorophenyl and the combination of B and Y^{m-} corresponds in each case to one row of table B (Compounds (IIa)-(1).9bB-1 to (IIa)-(1).9bB-440)

### Table 10b

Compounds (IIa)-(1), wherein A is 2-fluoro-6-chlorophenyl I and the combination of B and Y^{m-} corresponds in each case to one row of table B (Compounds (IIa)-(1).10b13-1 to (IIa)-(1).10bB-440)

### Table 11b

Compounds (IIa)-(1), wherein A is 3-fluoro-4-chlorophenyl and the combination of B and Y^{m-} corresponds in each case to one row of table B (Compounds (IIa)-(1).11bB-1 to (IIa)-(1).11bB-440)

### Table 12b

Compounds (IIa)-(1), wherein A is 3-fluoro-5-chlorophenyl and the combination of B and Y^{m-} corresponds in each case to one row of table B (Compounds (IIa)-(1).12bB-1 to (IIa)-(1).12bB-440)

### Table 13b

Compounds (IIa)-(1), wherein A is 2-chloro-3-fluorophenyl and the combination of B and Y^{m-} corresponds in each case to one row of table B (Compounds (IIa)-(1).13bB-1 to (IIa)-(1).13bB-440)

### Table 14b

Compounds (IIa)-(1), wherein A is 2-chloro-4-fluorophenyl and the combination of B and Y^{m-} corresponds in each case to one row of table B (Compounds (IIa)-(1).14bB-1 to (IIa)-(1).14bB-440)

### Table 15b

Compounds (IIa)-(1), wherein A is 2-chloro-5-fluorophenyl and the combination of B and Y^{m-} corresponds in each case to one row of table B (Compounds (IIa)-(1).15bB-1 to (IIa)-(1).15bB-440)

### Table 16b

Compounds (IIa)-(1), wherein A is 3-chloro-4-fluorophenyl and the combination of B and Y^{m-} corresponds in each case to one row of table B (Compounds (IIa)-(1).16bB-1 to (IIa)-(1).16bB-440)

### Table 17b

Compounds (IIa)-(1), wherein A is 2-methyl-3-fluorophenyl and the combination of B and Y^{m-} corresponds in each case to one row of table B (Compounds (IIa)-(1).17bB-1 to (IIa)-(1).17bB-440)

### Table 18b

Compounds (IIa)-(1), wherein A is 2-methyl-4-fluorophenyl and the combination of B and Y^{m-} corresponds in each case to one row of table B (Compounds (IIa)-(1).18bB-1 to (IIa)-(1).18bB-440)

### Table 19b

Compounds (IIa)-(1), wherein A is 2-methyl-5-fluorophenyl and the combination of B and Y^{m-} corresponds in each case to one row of table B (Compounds (IIa)-(1).19bB-1 to (IIa)-(1).19bB-440)

### Table 20b

Compounds (IIa)-(1), wherein A is 2-methyl-6-fluorophenyl and the combination of B and Y^{m-} corresponds in each case to one row of table B (Compounds (IIa)-(1).20bB-1 to (IIa)-(1).20bB-440)

### Table 21b

Compounds (IIa)-(1), wherein A is 3-methyl-4-fluorophenyl and the combination of B and Y^{m-} corresponds in each case to one row of table B (Compounds (IIa)-(1).21bB-1 to (IIa)-(1).21bB-440)

### Table 22b

Compounds (IIa)-(1), wherein A is 3-methyl-5-fluorophenyl and the combination of B and Y^{m-} corresponds in each case to one row of table B (Compounds (IIa)-(1).22bB-1 to (IIa)-(1).22bB-440)

### Table 23b

Compounds (IIa)-(1), wherein A is 2-fluoro-3-methylphenyl and the combination of B and Y^{m-} corresponds in each case to one row of table B (Compounds (IIa)-(1).23bB-1 to (IIa)-(1).23bB-440)

### Table 24b

Compounds (IIa)-(1), wherein A is 2-fluoro-4-methylphenyl and the combination of B and Y^{m-} corresponds in each case to one row of table B (Compounds (IIa)-(1).24bB-1 to (IIa)-(1).24bB-440)

### Table 25b

Compounds (IIa)-(1), wherein A is 2-fluoro-5-methylphenyl and the combination of B and Y^{m-} corresponds in each case to one row of table B (Compounds (IIa)-(1).25bB-1 to (IIa)-(1).25bB-440)

### Table 26b

Compounds (IIa)-(1), wherein A is 3-fluoro-4-methylphenyl and the combination of B and Y^{m-} corresponds in each case to one row of table B (Compounds (IIa)-(1).26bB-1 to (IIa)-(1).26bB-440)

### Table 27b

Compounds (IIa)-(1), wherein A is 2-methoxy-3-fluorophenyl and the combination of B and Y^{m-} corresponds in each case to one row of table B (Compounds (IIa)-(1).27bB-1 to (IIa)-(1).27bB-440)

### Table 28b

Compounds (IIa)-(1), wherein A is 2-methoxy-4-fluorophenyl and the combination of B and Y^{m-} corresponds in each case to one row of table B (Compounds (IIa)-(1).28bB-1 to (IIa)-(1).28bB-440)

### Table 29b

Compounds (IIa)-(1), wherein A is 2-methoxy-5-fluorophenyl and the combination of B and Y^{m-} corresponds in each case to one row of table B (Compounds (IIa)-(1).29bB-1 to (IIa)-(1).29bB-440)

### Table 30b

Compounds (IIa)-(1), wherein A is 2-methoxy-6-fluorophenyl and the combination of B and Y^{m-} corresponds in each case to one row of table B (Compounds (IIa)-(1).30bB-1 to (IIa)-(1).30bB-440)

### Table 31b

Compounds (IIa)-(1), wherein A is 3-methoxy-4-fluorophenyl and the combination of B and Y^{m-} corresponds in each case to one row of table B (Compounds (IIa)-(1).31bB-1 to (IIa)-(1).31bB-440)

### Table 32b

Compounds (IIa)-(1), wherein A is 3-methoxy-5-fluorophenyl and the combination of B and Y^{m-} corresponds in each case to one row of table B (Compounds (IIa)-(1).32bB-1 to (IIa)-(1).32bB-440)

### Table 33b

Compounds (IIa)-(1), wherein A is 2-fluoro-3-methoxyphenyl and the combination of B and Y^{m-} corresponds in each case to one row of table B (Compounds (IIa)-(1).33bB-1 to (IIa)-(1).33bB-440)

### Table 34b

Compounds (IIa)-(1), wherein A is 2-fluoro-4-methoxyphenyl and the combination of B and Y^{m-} corresponds in each case to one row of table B (Compounds (IIa)-(1).34bB-1 to (IIa)-(1).34bB-440)

### Table 35b

Compounds (IIa)-(1), wherein A is 2-fluoro-5-methoxyphenyl and the combination of B and Y^{m-} corresponds in each case to one row of table B (Compounds (IIa)-(1).35bB-1 to (IIa)-(1).35bB-440)

### Table 36b

Compounds (IIa)-(1), wherein A is 3-fluoro-4-methoxyphenyl and the combination of B and Y^{m-} corresponds in each case to one row of table B (Compounds (IIa)-(1).36bB-1 to (IIa)-(1).36bB-440)

### Table 37b

Compounds (IIa)-(1), wherein A is 3-fluoro-5-methoxyphenyl and the combination of B and Y^{m-} corresponds in each case to one row of table B (Compounds (IIa)-(1).37bB-1 to (IIa)-(1).37bB-440)

### Table 38b

Compounds (IIa)-(1), wherein A is 2-(difluoromethoxy)-3-fluorophenyl and the combination of B and Y^{m-} corresponds in each case to one row of table B (Compounds (IIa)-(1).38bB-1 to (IIa)-(1).38bB-440)

### Table 39b

Compounds (IIa)-(1), wherein A is 2-(difluoromethoxy)-4-fluorophenyl and the combination of B and Y^{m-} corresponds in each case to one row of table B (Compounds (IIa)-(1).39bB-1 to (IIa)-(1).39bB-440)

### Table 40b

Compounds (IIa)-(1), wherein A is 2-(difluoromethoxy)-5-fluorophenyl and the combination of B and Y^{m-} corresponds in each case to one row of table B (Compounds (IIa)-(1).40bB-1 to (IIa)-(1).40bB-440)

### Table 41b

Compounds (IIa)-(1), wherein A is 2-(difluoromethoxy)-6-fluorophenyl and the combination of B and Y^{m-} corresponds in each case to one row of table B (Compounds (IIa)-(1).41bB-1 to (IIa)-(1).41bB-440)

### Table 42b

Compounds (IIa)-(1), wherein A is 3-(difluoromethoxy)-4-fluorophenyl and the combination of B and Y^{m-} corresponds in each case to one row of table B (Compounds (IIa)-(1).42bB-1 to (IIa)-(1).42bB-440)

### Table 43b

Compounds (IIa)-(1), wherein A is 3-(difluoromethoxy)-5-fluorophenyl and the combination of B and Y^{m-}corresponds in each case to one row of table B (Compounds (IIa)-(1).73bB-1 to (IIa)-(1).73bB-440)

### Table 44b

Compounds (IIa)-(1), wherein A is 2-fluoro-3-(difluoromethoxy)phenyl and the combination of B and Y^{m-} corresponds in each case to one row of table B (Compounds (IIa)-(1).44bB-1 to (IIa)-(1).44bB-440)

### Table 45b

Compounds (IIa)-(1), wherein A is 2-fluoro-4-(difluoromethoxy)phenyl and the combination of B and Y^{m-}corresponds in each case to one row of table B (Compounds (IIa)-(1).45bB-1 to (IIa)-(1).45bB-440)

### Table 46b

Compounds (IIa)-(1), wherein A is 2-fluoro-5-(difluoromethoxy)phenyl and the combination of B and Y^{m-} corresponds in each case to one row of table B (Compounds (IIa)-(1).46bB-1 to (IIa)-(1).46bB-440)

### Table 47b

Compounds (IIa)-(1), wherein A is 3-fluoro-4-(difluoromethoxy)phenyl and the combination of B and Y^{m-} corresponds in each case to one row of table B (Compounds (IIa)-(1).47bB-1 to (IIa)-(1).47bB-440)

### Table 48b

Compounds (IIa)-(1), wherein A is 2,3,4-trifluorophenyl and the combination of B and Y^{m-} corresponds in each case to one row of table B (Compounds (IIa)-(1).48bB-1 to (IIa)-(1).48bB-440)

### Table 49b

Compounds (IIa)-(1), wherein A is 2,3,5-trifluorophenyl and the combination of B and Y^{m-} corresponds in each case to one row of table B (Compounds (IIa)-(1).49bB-1 to (IIa)-(1).49bB-440)

### Table 50b

Compounds (IIa)-(1), wherein A is 2,3,6-trifluorophenyl and the combination of B and Y^{m-} corresponds in each case to one row of table B (Compounds (IIa)-(1).50bB-1 to (IIa)-(1).50bB-440)

### Table 51b

Compounds (IIa)-(1), wherein A is 2,4,5-trifluorophenyl and the combination of B and Y^{m-} corresponds in each case to one row of table B (Compounds (IIa)-(1).51bB-1 to (IIa)-(1).51bB-440)

### Table 52b

Compounds (IIa)-(1), wherein A is 2,4,6-trifluorophenyl and the combination of B and Y^{m-} corresponds in each case to one row of table B (Compounds (IIa)-(1).52bB-1 to (IIa)-(1).52bB-440)

### Table 53b

Compounds (IIa)-(1), wherein A is 3,4,5-trifluorophenyl and the combination of B and Y^{m-} corresponds in each case to one row of table B (Compounds (IIa)-(1).53bB-1 to (IIa)-(1).53bB-440)

### Table 54b

Compounds (IIa)-(1), wherein A is phenyl and the combination of B and Y^{m-} corresponds in each case to one row of table B (Compounds (IIa)-(1).54bB-1 to (IIa)-(1).54bB-440)

### Table 55b

Compounds (IIa)-(1), wherein A is 2-chlorophenyl and the combination of B and Y^{m-} corresponds in each case to one row of table B (Compounds (IIa)-(1).55bB-1 to (IIa)-(1).55bB-440)

### Table 56b

Compounds (IIa)-(1), wherein A is 3-chlorophenyl and the combination of B and Y^{m-} corresponds in each case to one row of table B (Compounds (IIa)-(1).56bB-1 to (IIa)-(1).56bB-440)

### Table 57b

Compounds (IIa)-(1), wherein A is 4-chlorophenyl and the combination of B and Y^{m-} corresponds in each case to one row of table B (Compounds (IIa)-(1).57bB-1 to (IIa)-(1).57bB-440)

### Table 58b

Compounds (IIa)-(1), wherein A is 2-fluorophenyl and the combination of B and Y^{m-} corresponds in each case to one row of table B (Compounds (IIa)-(1).58bB-1 to (IIa)-(1).58bB-440)

### Table 59b

Compounds (IIa)-(1), wherein A is 3-fluorophenyl and the combination of B and Y^{m-} corresponds in each case to one row of table B (Compounds (IIa)-(1).59bB-1 to (IIa)-(1).59bB-440)

### Table 60b

Compounds (IIa)-(1), wherein A is 4-fluorophenyl and the combination of B and Y^{m-} corresponds in each case to one row of table B (Compounds (IIa)-(1).60bB-1 to (IIa)-(1).60bB-440)

### Table 61b

Compounds (IIa)-(1), wherein A is 2-methylphenyl and the combination of B and Y^{m-} corresponds in each case to one row of table B (Compounds (IIa)-(1).61bB-1 to (IIa)-(1).61bB-440)

### Table 62b v

Compounds (IIa)-(1), wherein A is 3-methylphenyl and the combination of B and Y^{m-} corresponds in each case to one row of table B (Compounds (IIa)-(1).62bB-1 to (IIa)-(1).62bB-440)

### Table 63b

Compounds (IIa)-(1), wherein A is 4-methylphenyl and the combination of B and Y^{m-} corresponds in each case to one row of table B (Compounds (IIa)-(1).63bB-1 to (IIa)-(1).63bB-440)

### Table 64b

Compounds (IIa)-(1), wherein A is 2-methoxyphenyl and the combination of B and Y^{m-} corresponds in each case to one row of table B (Compounds (IIa)-(1).64bB-1 to (IIa)-(1).64bB-440)

### Table 65b

Compounds (IIa)-(1), wherein A is 3-methoxyphenyl and the combination of B and

Y^{m-}corresponds in each case to one row of table B (Compounds (IIa)-(1).65bB-1 to (IIa)-(1).65bB-440)

### Table 66b

Compounds (IIa)-(1), wherein A is 4-methoxyphenyl and the combination of B and

Y^{m-} corresponds in each case to one row of table B (Compounds (IIa)-(1).66bB-1 to (IIa)-(1).66bB-440)

### Table 67b

Compounds (IIa)-(1), wherein A is 2-trifluoromethylphenyl and the combination of B and Y^{m-} corresponds in each case to one row of table B (Compounds (IIa)-(1).67bB-1 to (IIa)-(1).67bB-440)

### Table 68b

Compounds (IIa)-(1), wherein A is 3-trifluoromethylphenyl and the combination of B and Y^{m-} corresponds in each case to one row of table B (Compounds (IIa)-(1).68bB-1 to (IIa)-(1).68bB-440)

### Table 69b

Compounds (IIa)-(1), wherein A is 4-trifluoromethylphenyl and the combination of B and Y^{m-} corresponds in each case to one row of table B (Compounds (IIa)-(1).69bB-1 to (IIa)-(1).69bB-440)

### Table 70b

Compounds (IIa)-(1), wherein A is 2-difluoromethoxyphenyl and the combination of B and Y^{m-} corresponds in each case to one row of table B (Compounds (IIa)-(1).70bB-1 to (IIa)-(1).70bB-440)

### Table 71b

Compounds (IIa)-(1), wherein A is 3-difluoromethoxyphenyl and the combination of B and Y^{m-} corresponds in each case to one row of table B (Compounds (IIa)-(1).71bB-1 to (IIa)-(1).71bB-440)

### Table 72b

Compounds (IIa)-(1), wherein A is 4-difluoromethoxyphenyl and the combination of B and Y^{m-} corresponds in each case to one row of table B (Compounds (IIa)-(1).72bB-1 to (IIa)-(1).72bB-440)

### Table 73b

Compounds (IIa)-(1), wherein A is 2,3-dichlorophenyl and the combination of B and Y^{m-} corresponds in each case to one row of table B (Compounds (IIa)-(1).73bB-1 to (IIa)-(1).73bB-440)

### Table 74b

Compounds (IIa)-(1), wherein A is 2,4-dichlorophenyl and the combination of B and

Y^{m-} corresponds in each case to one row of table B (Compounds (IIa)-(1).74bB-1 to (IIa)-(1).74bB-440)

### Table 75b

Compounds (IIa)-(1), wherein A is 2,5-dichlorophenyl and the combination of B and Y^{m-} corresponds in each case to one row of table B (Compounds (IIa)-(1).75bB-1 to (IIa)-(1).75bB-440)

### Table 76b

Compounds (IIa)-(1), wherein A is 2,6-dichlorophenyl and the combination of B and Y^{m-} corresponds in each case to one row of table B (Compounds (IIa)-(1).76bB-1 to (IIa)-(1).76bB-440)

### Table 77b

Compounds (IIa)-(1), wherein A is 3,4-dichlorophenyl and the combination of B and

Y^{m-} corresponds in each case to one row of table B (Compounds (IIa)-(1).77bB-1 to (IIa)-(1).77bB-440)

### Table 78b

Compounds (IIa)-(1), wherein A is 3,5-dichlorophenyl and the combination of B and Y^{m-} corresponds in each case to one row of table B (Compounds (IIa)-(1).78bB-1 to (IIa)-(1).78bB-440)

### Table 79b

Compounds (IIa)-(1), wherein A is 2,3-dimethylphenyl and the combination of B and Y^{m-} corresponds in each case to one row of table B (Compounds (IIa)-(1).79bB-1 to (IIa)-(1).79bB-440)

### Table 80b

Compounds (IIa)-(1), wherein A is 2,4-dimethylphenyl and the combination of B and Y^{m-} corresponds in each case to one row of table B (Compounds (IIa)-(1).80bB-1 to (IIa)-(1).80bB-440)

### Table 81b

Compounds (IIa)-(1), wherein A is 2,5-dimethylphenyl and the combination of B and Y^{m-} corresponds in each case to one row of table B (Compounds (IIa)-(1).81bB-1 to (IIa)-(1).81bB-440)

### Table 82b

Compounds (IIa)-(1), wherein A is 2,6-dimethylphenyl and the combination of B and

Y^{m-} corresponds in each case to one row of table B (Compounds (IIa)-(1).82bB-1 to (IIa)-(1).82bB-440)

### Table 83b

Compounds (IIa)-(1), wherein A is 3,4-dimethylphenyl and the combination of B and Y^{m-} corresponds in each case to one row of table B (Compounds (IIa)-(1).83bB-1 to (IIa)-(1).83bB-440)

### Table 84b

Compounds (IIa)-(1), wherein A is 3,5-dimethylphenyl and the combination of B and Y^{m-} corresponds in each case to one row of table B (Compounds (IIa)-(1).84bB-1 to (IIa)-(1).84bB-440)

### Table 85b

Compounds (IIa)-(1), wherein A is 2,3-dimethoxyphenyl and the combination of B and Y^{m-} corresponds in each case to one row of table B (Compounds (IIa)-(1).85bB-1 to (IIa)-(1).85bB-440)

### Table 86b

Compounds (IIa)-(1), wherein A is 2,4-dimethoxyphenyl and the combination of B and Y^{m-} corresponds in each case to one row of table B (Compounds (IIa)-(1).86bB-1 to (IIa)-(1).861bB-440)

### Table 87b

Compounds (IIa)-(1), wherein A is 2,5-dimethoxyphenyl and the combination of B and Y^{m-} corresponds in each case to one row of table B (Compounds (IIa)-(1).87bB-1 to (IIa)-(1).87bB-440)

### Table 88b

Compounds (IIa)-(1), wherein A is 2,6-dimethoxyphenyl and the combination of B and Y^{m-} corresponds in each case to one row of table B (Compounds (IIa)-(1).88bB-1 to (IIa)-(1).88bB-440)

### Table 89b

Compounds (IIa)-(1), wherein A is 3,4-dimethoxyphenyl and the combination of B and Y^{m-} corresponds in each case to one row of table B (Compounds (IIa)-(1).89bB-1 to (IIa)-(1).89bB-440)

### Table 90b

Compounds (IIa)-(1), wherein A is 3,5-dimethoxyphenyl and the combination of B and Y^{m-} corresponds in each case to one row of table B (Compounds (IIa)-(1).90bB-1 to (IIa)-(1).90bB-440)

### Table 91b

Compounds (IIa)-(1), wherein A is 2-methyl-3-chlorophenyl and the combination of B and Y^{m-} corresponds in each case to one row of table B (Compounds (IIa)-(1).91bB-1 to (IIa)-(1).91bB-440)

### Table 92b

Compounds (IIa)-(1), wherein A is 2-methyl-4-chlorophenyl and the combination of B and Y^{m-} corresponds in each case to one row of table B (Compounds (IIa)-(1).92bB-1 to (IIa)-(1).92bB-440)

### Table 93b

Compounds (IIa)-(1), wherein A is 2-methyl-5-chlorophenyl and the combination of B and Y^{m-} corresponds in each case to one row of table B (Compounds (IIa)-(1).93bB-1 to (IIa)-(1).93bB-440)

### Table 94b

Compounds (IIa)-(1), wherein A is 2-methyl-6-chlorophenyl and the combination of B and Y^{m-} corresponds in each case to one row of table B (Compounds (IIa)-(1).94bB-1 to (IIa)-(1).94bB3-440)

### Table 95b

Compounds (IIa)-(1), wherein A is 3-methyl-4-chlorophenyl and the combination of B and Y^{m-} corresponds in each case to one row of table B (Compounds (IIa)-(1).95bB-1 to (IIa)-(1).95bB-440)

### Table 96b

Compounds (IIa)-(1), wherein A is 3-methyl-5-chlorophenyl and the combination of B and Y^{m-} corresponds in each case to one row of table B (Compounds (IIa)-(1).96bB-1 to (IIa)-(1).96bB-440)

### Table 97b

Compounds (IIa)-(1), wherein A is 2-chloro-3-methylphenyl and the combination of B and Y^{m-} corresponds in each case to one row of table B (Compounds (IIa)-(1).97bB-1 to (IIa)-(1).97bB-440)

### Table 98b

Compounds (IIa)-(1), wherein A is 2-chloro-4-methylphenyl and the combination of

B and Y^{m-} corresponds in each case to one row of table B (Compounds (IIa)-(1).98bB-1 to (IIa)-(1).98bB-440)

### Table 99b

Compounds (IIa)-(1), wherein A is 2-chloro-5-methylphenyl and the combination of B and Y^{m-} corresponds in each case to one row of table B (Compounds (IIa)-(1).99bB-1 to (IIa)-(1).99bB-440)

### Table 100b

Compounds (IIa)-(1), wherein A is 3-chloro-4-methylphenyl and the combination of B and Y^{m-} corresponds in each case to one row of table B (Compounds (IIa)-(1).100bB-1 to (IIa)-(1).100bB-440)

### Table 101b

Compounds (IIa)-(1), wherein A is 2-methoxy-3-chlorophenyl and the combination of B and Y^{m-} corresponds in each case to one row of table B (Compounds (IIa)-(1).101bB-1 to (IIa)-(1).101bB-440)

### Table 102b

Compounds (IIa)-(1), wherein A is 2-methoxy-4-chlorophenyl and the combination of B and Y^{m-} corresponds in each case to one row of table B (Compounds (IIa)-(1).102bB-1 to (IIa)-(1).102bB-440)

### Table 103b

Compounds (IIa)-(1), wherein A is 2-methoxy-5-chlorophenyl and the combination of B and Y^{m-} corresponds in each case to one row of table B (Compounds (IIa)-(1).103bB-1 to (IIa)-(1).103bB-440)

### Table 104b

Compounds (IIa)-(1), wherein A is 2-methoxy-6-chlorophenyl and the combination of B and Y^{m-} corresponds in each case to one row of table B (Compounds (IIa)-(1).104bB-1 to (IIa)-(1).104bB-440)

### Table 105b

Compounds (IIa)-(1), wherein A is 3-methoxy-4-chlorophenyl and the combination of B and Y^{m-} corresponds in each case to one row of table B (Compounds (IIa)-(1).105bB-1 to (IIa)-(1).105bB-440)

### Table 106b

Compounds (IIa)-(1), wherein A is 3-methoxy-5-chlorophenyl and the combination of B and Y^{m-} corresponds in each case to one row of table B (Compounds (IIa)-(1).106bB-1 to (IIa)-(1).106bB-440)

### Table 107b

Compounds (IIa)-(1), wherein A is 2-chloro-3-methoxyphenyl and the combination of B and Y^{m-} corresponds in each case to one row of table B (Compounds (IIa)-(1).107bB-1 to (IIa)-(1).107bB-440)

### Table 108b

Compounds (IIa)-(1), wherein A is 2-chloro-4-methoxyphenyl and the combination of B and Y^{m-} corresponds in each case to one row of table B (Compounds (IIa)-(1).108bB-1 to (IIa)-(1).108bB-440)

### Table 109b

Compounds (IIa)-(1), wherein A is 2-chloro-5-methoxyphenyl and the combination of B and Y^{m-} corresponds in each case to one row of table B (Compounds (IIa)-(1).109bB-1 to (IIa)-(1).109bB-440)

### Table 110b

Compounds (IIa)-(1), wherein A is 3-chloro-4-methoxyphenyl and the combination of B and Y^{m-} corresponds in each case to one row of table B (Compounds (IIa)-(1).110bB-1 to (IIa)-(1).110bB-440)

### Table 111b

Compounds (IIa)-(1), wherein A is 2-(trifluoromethyl)-3-chlorophenyl and the combination of B and Y^{m-} corresponds in each case to one row of table B (Compounds (IIa)-(1).111bB-1 to (IIa)-(1).111bB-440)

### Table 112b

Compounds (IIa)-(1), wherein A is 2-(trifluoromethyl)-4-chlorophenyl and the combination of B and Y^{m-} corresponds in each case to one row of table B (Compounds (IIa)-(1).112bB-1 to (IIa)-(1).112bB-440)

### Table 113b

Compounds (IIa)-(1), wherein A is 2-(trifluoromethyl)-5-chlorophenyl and the combination of B and Y^{m-} corresponds in each case to one row of table B (Compounds (IIa)-(1).113bB-1 to (IIa)-(1).113bB-440)

### Table 114b

Compounds (IIa)-(1), wherein A is 2-(trifluoromethyl)-6-chlorophenyl and the combination of B and Y^{m-} corresponds in each case to one row of table B (Compounds (IIa)-(1).114bB-1 to (IIa)-(1).114bB-440)

### Table 115b

Compounds (IIa)-(1), wherein A is 3-(trifluoromethyl)-4-chlorophenyl and the combination of B and Y^{m-} corresponds in each case to one row of table B (Compounds (IIa)-(1).115bB-1 to (IIa)-(1).115bB-440)

### Table 116b

Compounds (IIa)-(1), wherein A is 3-(trifluoromethyl)-5-chlorophenyl and the combination of B and Y^{m-} corresponds in each case to one row of table B (Compounds (IIa)-(1).116bB-1 to (IIa)-(1).116bB-440)

### Table 117b

Compounds (IIa)-(1), wherein A is 2-chloro-3-(trifluoromethyl)phenyl and the combination of B and Y^{m-} corresponds in each case to one row of table B (Compounds (IIa)-(1).117bB-1 to (IIa)-(1).117bB-440)

### Table 118b

Compounds (IIa)-(1), wherein A is 2-chloro-4-(trifluoromethyl)phenyl and the combination of B and Y^{m-} corresponds in each case to one row of table B (Compounds (IIa)-(1).118bB-1 to (IIa)-(1).118bB-440)

### Table 119b

Compounds (IIa)-(1), wherein A is 2-chloro-5-(trifluoromethyl)phenyl and the combination of B and Y^{m-} corresponds in each case to one row of table B (Compounds (IIa)-(1).119bB-1 to (IIa)-(1).119bB-440)

### Table 120b

Compounds (IIa)-(1), wherein A is 3-chloro-4-(trifluoromethyl)phenyl and the combination of B and Y^{m-} corresponds in each case to one row of table B (Compounds (IIa)-(1).120bB-1 to (IIa)-(1).120bB-440)

### Table 121b

Compounds (IIa)-(1), wherein A is 2-(trifluoromethoxy)-3-chlorophenyl and the combination of B and Y^{m-} corresponds in each case to one row of table B (Corn-pounds (IIa)-(1).121bB-1 to (IIa)-(1).121bB-440)

### Table 122b

Compounds (IIa)-(1), wherein A is 2-(difluoromethoxy)-3-chlorophenyl and the combination of B and Y^{m-} corresponds in each case to one row of table B (Compounds (IIa)-(1).122bB-1 to (IIa)-(1).122bB-440)

### Table 123b

Compounds (IIa)-(1), wherein A is 2-(difluoromethoxy)-4-chlorophenyl and the combination of B and Y^{m-} corresponds in each case to one row of table B (Compounds (IIa)-(1).123bB-1 to (IIa)-(1).123bB-440)

### Table 124b

Compounds (IIa)-(1), wherein A is 2-(difluoromethoxy)-5-chlorophenyl and the combination of B and Y^{m-} corresponds in each case to one row of table B (Compounds (IIa)-(1).124bB-1 to (IIa)-(1).124bB-440)

### Table 125b

Compounds (IIa)-(1), wherein A is 2-(difluoromethoxy)-6-chlorophenyl and the combination of B and Y^{m-} corresponds in each case to one row of table B (Compounds (IIa)-(1).125bB-1 to (IIa)-(1).125bB-440)

### Table 126b

Compounds (IIa)-(1), wherein A is 3-(difluoromethoxy)-4-chlorophenyl and the combination of B and Y^{m-} corresponds in each case to one row of table B (Compounds (IIa)-(1).126bB-1 to (IIa)-(1).126bB-440)

### Table 127b

Compounds (IIa)-(1), wherein A is 3-(difluoromethoxy)-5-chlorophenyl and the combination of B and Y^{m-} corresponds in each case to one row of table B (Compounds (IIa)-(1).127bB-1 to (IIa)-(1).127bB-440)

### Table 128b

Compounds (IIa)-(1), wherein A is 2-chloro-3-(difluoromethoxy)phenyl and the combination of B and Y^{m-} corresponds in each case to one row of table B (Compounds (IIa)-(1).128bB-1 to (IIa)-(1).128bB-440)

### Table 129b

Compounds (IIa)-(1), wherein A is 2-chloro-4-(difluoromethoxy)phenyl and the combination of B and Y^{m-} corresponds in each case to one row of table B (Compounds (IIa)-(1).129bB-1 to (IIa)-(1).129bB-440)

### Table 130b

Compounds (IIa)-(1), wherein A is 2-chloro-5-(difluoromethoxy)phenyl and the combination of B and Y^{m-} corresponds in each case to one row of table B (Compounds (IIa)-(1).130bB-1 to (IIa)-(1).130bB-440)

### Table 131b

Compounds (IIa)-(1), wherein A is 3-chloro-4-(difluoromethoxy)phenyl and the combination of B and Y^{m-}corresponds in each case to one row of table B (Compounds (IIa)-(1).131bB-1 to (IIa)-(1).131bB-440)

### Table 132b

Compounds (IIa)-(1), wherein A is 2,3,4-trichlorophenyl and the combination of B and Y^{m-} corresponds in each case to one row of table B (Compounds (IIa)-(1).132bB-1 to (IIa)-(1).132bB-440)

### Table 133b

Compounds (IIa)-(1), wherein A is 2,3,5-trichlorophenyl and the combination of B and Y^{m-} corresponds in each case to one row of table B (Compounds (IIa)-(1).133bB-1 to (IIa)-(1).133bB-440)

### Table 134b

Compounds (IIa)-(1), wherein A is 2,3,6-trichlorophenyl and the combination of B and Y^{m-} corresponds in each case to one row of table B (Compounds (IIa)-(1).134bB-1 to (IIa)-(1).134bB-440)

### Table 135b

Compounds (IIa)-(1), wherein A is 2,4,5-trichlorophenyl and the combination of B and Y^{m-} corresponds in each case to one row of table B (Compounds (IIa)-(1).135bB-1 to (IIa)-(1).135bB-440)

### Table 136b

Compounds (IIa)-(1), wherein A is 2,4,6-trichlorophenyl and the combination of B and Y^{m-} corresponds in each case to one row of table B (Compounds (IIa)-(1).136bB-1 to (IIa)-(1).136bB-440)

### Table 137b

Compounds (IIa)-(1), wherein A is 3,4,5-trichlorophenyl and the combination of B and Y^{m-} corresponds in each case to one row of table B (Compounds (IIa)-(1).137bB-1 to (IIa)-(1).137bB-440)

TABLE B

| row | B | Y^{m-} |
|---|---|---|
| B-1 | phenyl | Cl⁻ |
| B-2 | 2-chlorophenyl | Cl⁻ |
| B-3 | 3-chlorophenyl | Cl⁻ |
| B-4 | 4-chlorophenyl | Cl⁻ |
| B-5 | 2-fluorophenyl | Cl⁻ |
| B-6 | 3-fluorophenyl | Cl⁻ |
| B-7 | 4-fluorophenyl | Cl⁻ |
| B-8 | 2-methylphenyl | Cl⁻ |
| B-9 | 3-methylphenyl | Cl⁻ |
| B-10 | 4-methylphenyl | Cl⁻ |
| B-11 | 2-methoxyphenyl | Cl⁻ |
| B-12 | 3-methoxyphenyl | Cl⁻ |
| B-13 | 4-methoxyphenyl | Cl⁻ |
| B-14 | 2,3-dichlorophenyl | Cl⁻ |
| B-15 | 2,4-dichlorophenyl | Cl⁻ |
| B-16 | 2,5-dichlorophenyl | Cl⁻ |
| B-17 | 2,6-dichlorophenyl | Cl⁻ |
| B-18 | 3,4-dichlorophenyl | Cl⁻ |
| B-19 | 3,5-dichlorophenyl | Cl⁻ |
| B-20 | 2,3-difluorophenyl | Cl⁻ |
| B-21 | 2,4-difluorophenyl | Cl⁻ |
| B-22 | 2,5-difluorophenyl | Cl⁻ |
| B-23 | 2,6-difluorophenyl | Cl⁻ |
| B-24 | 3,4-difluorophenyl | Cl⁻ |
| B-25 | 3,5-difluorophenyl | Cl⁻ |
| B-26 | 2-fluoro-3-chlorophenyl | Cl⁻ |
| B-27 | 2-fluoro-4-chlorophenyl | Cl⁻ |
| B-28 | 2-fluoro-5-chlorophenyl | Cl⁻ |
| B-29 | 2-fluoro-6-chlorophenyl | Cl⁻ |
| B-30 | 3-fluoro-4-chlorophenyl | Cl⁻ |
| B-31 | 3-fluoro-5-chlorophenyl | Cl⁻ |
| B-32 | 2-chloro-3-fluorophenyl | Cl⁻ |
| B-33 | 2-chloro-4-fluorophenyl | Cl⁻ |
| B-34 | 2-chloro-5-fluorophenyl | Cl⁻ |
| B-35 | 3-chloro-4-fluorophenyl | Cl⁻ |
| B-36 | 2-methyl-3-chlorophenyl | Cl⁻ |
| B-37 | 2-methyl-4-chlorophenyl | Cl⁻ |
| B-38 | 2-methyl-5-chlorophenyl | Cl⁻ |
| B-39 | 2-methyl-6-chlorophenyl | Cl⁻ |
| B-40 | 3-methyl-4-chlorophenyl | Cl⁻ |
| B-41 | 3-methyl-5-chlorophenyl | Cl⁻ |
| B-42 | 2-chloro-3-methylphenyl | Cl⁻ |
| B-43 | 2-chloro-4-methylphenyl | Cl⁻ |
| B-44 | 2-chloro-5-methylphenyl | Cl⁻ |
| B-45 | 3-chloro-4-methylphenyl | Cl⁻ |
| B-46 | 2-methyl-3-fluorophenyl | Cl⁻ |
| B-47 | 2-methyl-4-fluorophenyl | Cl⁻ |
| B-48 | 2-methyl-5-fluorophenyl | Cl⁻ |
| B-49 | 2-methyl-6-fluorophenyl | Cl⁻ |
| B-50 | 3-methyl-4-fluorophenyl | Cl⁻ |
| B-51 | 3-methyl-5-fluorophenyl | Cl⁻ |
| B-52 | 2-fluoro-3-methylphenyl | Cl⁻ |
| B-53 | 2-fluoro-4-methylphenyl | Cl⁻ |
| B-54 | 2-fluoro-5-methylphenyl | Cl⁻ |
| B-55 | 3-fluoro-4-methylphenyl | Cl⁻ |
| B-56 | phenyl | SO₄²⁻ |
| B-57 | 2-chlorophenyl | SO₄²⁻ |
| B-58 | 3-chlorophenyl | SO₄²⁻ |
| B-59 | 4-chlorophenyl | SO₄²⁻ |
| B-60 | 2-fluorophenyl | SO₄²⁻ |
| B-61 | 3-fluorophenyl | SO₄²⁻ |
| B-62 | 4-fluorophenyl | SO₄²⁻ |
| B-63 | 2-methylphenyl | SO₄²⁻ |
| B-64 | 3-methylphenyl | SO₄²⁻ |
| B-65 | 4-methylphenyl | SO₄²⁻ |
| B-66 | 2-methoxyphenyl | SO₄²⁻ |
| B-67 | 3-methoxyphenyl | SO₄²⁻ |
| B-68 | 4-methoxyphenyl | SO₄²⁻ |
| B-69 | 2,3-dichlorophenyl | SO₄²⁻ |
| B-70 | 2,4-dichlorophenyl | SO₄²⁻ |
| B-71 | 2,5-dichlorophenyl | SO₄²⁻ |
| B-72 | 2,6-dichlorophenyl | SO₄²⁻ |
| B-73 | 3,4-dichlorophenyl | SO₄²⁻ |
| B-74 | 3,5-dichlorophenyl | SO₄²⁻ |
| B-75 | 2,3-difluorophenyl | SO₄²⁻ |
| B-76 | 2,4-difluorophenyl | SO₄²⁻ |
| B-77 | 2,5-difluorophenyl | SO₄²⁻ |
| B-78 | 2,6-difluorophenyl | SO₄²⁻ |
| B-79 | 3,4-difluorophenyl | SO₄²⁻ |
| B-80 | 3,5-difluorophenyl | SO₄²⁻ |
| B-81 | 2-fluoro-3-chlorophenyl | SO₄²⁻ |
| B-82 | 2-fluoro-4-chlorophenyl | SO₄²⁻ |
| B-83 | 2-fluoro-5-chlorophenyl | SO₄²⁻ |
| B-84 | 2-fluoro-6-chlorophenyl | SO₄²⁻ |
| B-85 | 3-fluoro-4-chlorophenyl | SO₄²⁻ |
| B-86 | 3-fluoro-5-chlorophenyl | SO₄²⁻ |
| B-87 | 2-chloro-3-fluorophenyl | SO₄²⁻ |
| B-88 | 2-chloro-4-fluorophenyl | SO₄²⁻ |
| B-89 | 2-chloro-5-fluorophenyl | SO₄²⁻ |
| B-90 | 3-chloro-4-fluorophenyl | SO₄²⁻ |
| B-91 | 2-methyl-3-chlorophenyl | SO₄²⁻ |
| B-92 | 2-methyl-4-chlorophenyl | SO₄²⁻ |
| B-93 | 2-methyl-5-chlorophenyl | SO₄²⁻ |
| B-94 | 2-methyl-6-chlorophenyl | SO₄²⁻ |
| B-95 | 3-methyl-4-chlorophenyl | SO₄²⁻ |
| B-96 | 3-methyl-5-chlorophenyl | SO₄²⁻ |
| B-97 | 2-chloro-3-methylphenyl | SO₄²⁻ |
| B-98 | 2-chloro-4-methylphenyl | SO₄²⁻ |
| B-99 | 2-chloro-5-methylphenyl | SO₄²⁻ |
| B-100 | 3-chloro-4-methylphenyl | SO₄²⁻ |
| B-101 | 2-methyl-3-fluorophenyl | SO₄²⁻ |
| B-102 | 2-methyl-4-fluorophenyl | SO₄²⁻ |
| B-103 | 2-methyl-5-fluorophenyl | SO₄²⁻ |
| B-104 | 2-methyl-6-fluorophenyl | SO₄²⁻ |
| B-105 | 3-methyl-4-fluorophenyl | SO₄²⁻ |
| B-106 | 3-methyl-5-fluorophenyl | SO₄²⁻ |
| B-107 | 2-fluoro-3-methylphenyl | SO₄²⁻ |
| B-108 | 2-fluoro-4-methylphenyl | SO₄²⁻ |
| B-109 | 2-fluoro-5-methylphenyl | SO₄²⁻ |
| B-110 | 3-fluoro-4-methylphenyl | SO₄²⁻ |
| B-111 | phenyl | HSO₄⁻ |
| B-112 | 2-chlorophenyl | HSO₄⁻ |
| B-113 | 3-chlorophenyl | HSO₄⁻ |
| B-114 | 4-chlorophenyl | HSO₄⁻ |
| B-115 | 2-fluorophenyl | HSO₄⁻ |
| B-116 | 3-fluorophenyl | HSO₄⁻ |
| B-117 | 4-fluorophenyl | HSO₄⁻ |
| B-118 | 2-methylphenyl | HSO₄⁻ |
| B-119 | 3-methylphenyl | HSO₄⁻ |
| B-120 | 4-methylphenyl | HSO₄⁻ |
| B-121 | 2-methoxyphenyl | HSO₄⁻ |
| B-122 | 3-methoxyphenyl | HSO₄⁻ |
| B-123 | 4-methoxyphenyl | HSO₄⁻ |
| B-124 | 2,3-dichlorophenyl | HSO₄⁻ |
| B-125 | 2,4-dichlorophenyl | HSO₄⁻ |
| B-126 | 2,5-dichlorophenyl | HSO₄⁻ |
| B-127 | 2,6-dichlorophenyl | HSO₄⁻ |
| B-128 | 3,4-dichlorophenyl | HSO₄⁻ |
| B-129 | 3,5-dichlorophenyl | HSO₄⁻ |
| B-130 | 2,3-difluorophenyl | HSO₄⁻ |
| B-131 | 2,4-difluorophenyl | HSO₄⁻ |
| B-132 | 2,5-difluorophenyl | HSO₄⁻ |
| B-133 | 2,6-difluorophenyl | HSO₄⁻ |
| B-134 | 3,4-difluorophenyl | HSO₄⁻ |
| B-135 | 3,5-difluorophenyl | HSO₄⁻ |
| B-136 | 2-fluoro-3-chlorophenyl | HSO₄⁻ |
| B-137 | 2-fluoro-4-chlorophenyl | HSO₄⁻ |
| B-138 | 2-fluoro-5-chlorophenyl | HSO₄⁻ |
| B-139 | 2-fluoro-6-chlorophenyl | HSO₄⁻ |
| B-140 | 3-fluoro-4-chlorophenyl | HSO₄⁻ |
| B-141 | 3-fluoro-5-chlorophenyl | HSO₄⁻ |
| B-142 | 2-chloro-3-fluorophenyl | HSO₄⁻ |
| B-143 | 2-chloro-4-fluorophenyl | HSO₄⁻ |
| B-144 | 2-chloro-5-fluorophenyl | HSO₄⁻ |
| B-145 | 3-chloro-4-fluorophenyl | HSO₄⁻ |
| B-146 | 2-methyl-3-chlorophenyl | HSO₄⁻ |
| B-147 | 2-methyl-4-chlorophenyl | HSO₄⁻ |
| B-148 | 2-methyl-5-chlorophenyl | HSO₄⁻ |
| B-149 | 2-methyl-6-chlorophenyl | HSO₄⁻ |
| B-150 | 3-methyl-4-chlorophenyl | HSO₄⁻ |
| B-151 | 3-methyl-5-chlorophenyl | HSO₄⁻ |
| B-152 | 2-chloro-3-methylphenyl | HSO₄⁻ |
| B-153 | 2-chloro-4-methylphenyl | HSO₄⁻ |
| B-154 | 2-chloro-5-methylphenyl | HSO₄⁻ |
| B-155 | 3-chloro-4-methylphenyl | HSO₄⁻ |
| B-156 | 2-methyl-3-fluorophenyl | HSO₄⁻ |
| B-157 | 2-methyl-4-fluorophenyl | HSO₄⁻ |
| B-158 | 2-methyl-5-fluorophenyl | HSO₄⁻ |
| B-159 | 2-methyl-6-fluorophenyl | HSO₄⁻ |
| B-160 | 3-methyl-4-fluorophenyl | HSO₄⁻ |
| B-161 | 3-methyl-5-fluorophenyl | HSO₄⁻ |
| B-162 | 2-fluoro-3-methylphenyl | HSO₄⁻ |
| B-163 | 2-fluoro-4-methylphenyl | HSO₄⁻ |
| B-164 | 2-fluoro-5-methylphenyl | HSO₄⁻ |
| B-165 | 3-fluoro-4-methylphenyl | HSO₄⁻ |
| B-166 | phenyl | PO₄³⁻ |
| B-167 | 2-chlorophenyl | PO₄³⁻ |
| B-168 | 3-chlorophenyl | PO₄³⁻ |
| B-169 | 4-chlorophenyl | PO₄³⁻ |
| B-170 | 2-fluorophenyl | PO₄³⁻ |
| B-171 | 3-fluorophenyl | PO₄³⁻ |
| B-172 | 4-fluorophenyl | PO₄³⁻ |
| B-173 | 2-methylphenyl | PO₄³⁻ |
| B-174 | 3-methylphenyl | PO₄³⁻ |
| B-175 | 4-methylphenyl | PO₄³⁻ |
| B-176 | 2-methoxyphenyl | PO₄³⁻ |
| B-177 | 3-methoxyphenyl | PO₄³⁻ |
| B-178 | 4-methoxyphenyl | PO₄³⁻ |
| B-179 | 2,3-dichlorophenyl | PO₄³⁻ |
| B-180 | 2,4-dichlorophenyl | PO₄³⁻ |
| B-181 | 2,5-dichlorophenyl | PO₄³⁻ |
| B-182 | 2,6-dichlorophenyl | PO₄³⁻ |
| B-183 | 3,4-dichlorophenyl | PO₄³⁻ |
| B-184 | 3,5-dichlorophenyl | PO₄³⁻ |
| B-185 | 2,3-difluorophenyl | PO₄³⁻ |
| B-186 | 2,4-difluorophenyl | PO₄³⁻ |
| B-187 | 2,5-difluorophenyl | PO₄³⁻ |
| B-188 | 2,6-difluorophenyl | PO₄³⁻ |
| B-189 | 3,4-difluorophenyl | PO₄³⁻ |
| B-190 | 3,5-difluorophenyl | PO₄³⁻ |
| B-191 | 2-fluoro-3-chlorophenyl | PO₄³⁻ |
| B-192 | 2-fluoro-4-chlorophenyl | PO₄³⁻ |
| B-193 | 2-fluoro-5-chlorophenyl | PO₄³⁻ |
| B-194 | 2-fluoro-6-chlorophenyl | PO₄³⁻ |
| B-195 | 3-fluoro-4-chlorophenyl | PO₄³⁻ |
| B-196 | 3-fluoro-5-chlorophenyl | PO₄³⁻ |
| B-197 | 2-chloro-3-fluorophenyl | PO₄³⁻ |
| B-198 | 2-chloro-4-fluorophenyl | PO₄³⁻ |
| B-199 | 2-chloro-5-fluorophenyl | PO₄³⁻ |
| B-200 | 3-chloro-4-fluorophenyl | PO₄³⁻ |
| B-201 | 2-methyl-3-chlorophenyl | PO₄³⁻ |
| B-202 | 2-methyl-4-chlorophenyl | PO₄³⁻ |
| B-203 | 2-methyl-5-chlorophenyl | PO₄³⁻ |
| B-204 | 2-methyl-6-chlorophenyl | PO₄³⁻ |
| B-205 | 3-methyl-4-chlorophenyl | PO₄³⁻ |
| B-206 | 3-methyl-5-chlorophenyl | PO₄³⁻ |
| B-207 | 2-chloro-3-methylphenyl | PO₄³⁻ |
| B-208 | 2-chloro-4-methylphenyl | PO₄³⁻ |
| B-209 | 2-chloro-5-methylphenyl | PO₄³⁻ |
| B-210 | 3-chloro-4-methylphenyl | PO₄³⁻ |
| B-211 | 2-methyl-3-fluorophenyl | PO₄³⁻ |
| B-212 | 2-methyl-4-fluorophenyl | PO₄³⁻ |
| B-213 | 2-methyl-5-fluorophenyl | PO₄³⁻ |
| B-214 | 2-methyl-6-fluorophenyl | PO₄³⁻ |
| B-215 | 3-methyl-4-fluorophenyl | PO₄³⁻ |
| B-216 | 3-methyl-5-fluorophenyl | PO₄³⁻ |
| B-217 | 2-fluoro-3-methylphenyl | PO₄³⁻ |
| B-218 | 2-fluoro-4-methylphenyl | PO₄³⁻ |
| B-219 | 2-fluoro-5-methylphenyl | PO₄³⁻ |
| B-220 | 3-fluoro-4-methylphenyl | PO₄³⁻ |
| B-221 | phenyl | HPO₄²⁻ |
| B-222 | 2-chlorophenyl | HPO₄²⁻ |
| B-223 | 3-chlorophenyl | HPO₄²⁻ |
| B-224 | 4-chlorophenyl | HPO₄²⁻ |
| B-225 | 2-fluorophenyl | HPO₄²⁻ |
| B-226 | 3-fluorophenyl | HPO₄²⁻ |
| B-227 | 4-fluorophenyl | HPO₄²⁻ |
| B-228 | 2-methylphenyl | HPO₄²⁻ |
| B-229 | 3-methylphenyl | HPO₄²⁻ |
| B-230 | 4-methylphenyl | HPO₄²⁻ |
| B-231 | 2-methoxyphenyl | HPO₄²⁻ |
| B-232 | 3-methoxyphenyl | HPO₄²⁻ |
| B-233 | 4-methoxyphenyl | HPO₄²⁻ |
| B-234 | 2,3-dichlorophenyl | HPO₄²⁻ |
| B-235 | 2,4-dichlorophenyl | HPO₄²⁻ |
| B-236 | 2,5-dichlorophenyl | HPO₄²⁻ |
| B-237 | 2,6-dichlorophenyl | HPO₄²⁻ |
| B-238 | 3,4-dichlorophenyl | HPO₄²⁻ |
| B-239 | 3,5-dichlorophenyl | HPO₄²⁻ |
| B-240 | 2,3-difluorophenyl | HPO₄²⁻ |
| B-241 | 2,4-difluorophenyl | HPO₄²⁻ |
| B-242 | 2,5-difluorophenyl | HPO₄²⁻ |
| B-243 | 2,6-difluorophenyl | HPO₄²⁻ |
| B-244 | 3,4-difluorophenyl | HPO₄²⁻ |
| B-245 | 3,5-difluorophenyl | HPO₄²⁻ |
| B-246 | 2-fluoro-3-chlorophenyl | HPO₄²⁻ |
| B-247 | 2-fluoro-4-chlorophenyl | HPO₄²⁻ |
| B-248 | 2-fluoro-5-chlorophenyl | HPO₄²⁻ |
| B-249 | 2-fluoro-6-chlorophenyl | HPO₄²⁻ |
| B-250 | 3-fluoro-4-chlorophenyl | HPO₄²⁻ |
| B-251 | 3-fluoro-5-chlorophenyl | HPO₄²⁻ |
| B-252 | 2-chloro-3-fluorophenyl | HPO₄²⁻ |
| B-253 | 2-chloro-4-fluorophenyl | HPO₄²⁻ |
| B-254 | 2-chloro-5-fluorophenyl | HPO₄²⁻ |
| B-255 | 3-chloro-4-fluorophenyl | HPO₄²⁻ |
| B-256 | 2-methyl-3-chlorophenyl | HPO₄²⁻ |
| B-257 | 2-methyl-4-chlorophenyl | HPO₄²⁻ |
| B-258 | 2-methyl-5-chlorophenyl | HPO₄²⁻ |
| B-259 | 2-methyl-6-chlorophenyl | HPO₄²⁻ |
| B-260 | 3-methyl-4-chlorophenyl | HPO₄²⁻ |
| B-261 | 3-methyl-5-chlorophenyl | HPO₄²⁻ |
| B-262 | 2-chloro-3-methylphenyl | HPO₄²⁻ |
| B-263 | 2-chloro-4-methylphenyl | HPO₄²⁻ |
| B-264 | 2-chloro-5-methylphenyl | HPO₄²⁻ |
| B-265 | 3-chloro-4-methylphenyl | HPO₄²⁻ |
| B-266 | 2-methyl-3-fluorophenyl | HPO₄²⁻ |
| B-267 | 2-methyl-4-fluorophenyl | HPO₄²⁻ |
| B-268 | 2-methyl-5-fluorophenyl | HPO₄²⁻ |
| B-269 | 2-methyl-6-fluorophenyl | HPO₄²⁻ |
| B-270 | 3-methyl-4-fluorophenyl | HPO₄²⁻ |
| B-271 | 3-methyl-5-fluorophenyl | HPO₄²⁻ |
| B-272 | 2-fluoro-3-methylphenyl | HPO₄²⁻ |
| B-273 | 2-fluoro-4-methylphenyl | HPO₄²⁻ |
| B-274 | 2-fluoro-5-methylphenyl | HPO₄²⁻ |
| B-275 | 3-fluoro-4-methylphenyl | HPO₄²⁻ |
| B-276 | phenyl | H₂PO₄⁻ |
| B-277 | 2-chlorophenyl | H₂PO₄⁻ |
| B-278 | 3-chlorophenyl | H₂PO₄⁻ |
| B-279 | 4-chlorophenyl | H₂PO₄⁻ |
| B-280 | 2-fluorophenyl | H₂PO₄⁻ |
| B-281 | 3-fluorophenyl | H₂PO₄⁻ |
| B-282 | 4-fluorophenyl | H₂PO₄⁻ |
| B-283 | 2-methylphenyl | H₂PO₄⁻ |
| B-284 | 3-methylphenyl | H₂PO₄⁻ |
| B-285 | 4-methylphenyl | H₂PO₄⁻ |
| B-286 | 2-methoxyphenyl | H₂PO₄⁻ |
| B-287 | 3-methoxyphenyl | H₂PO₄⁻ |
| B-288 | 4-methoxyphenyl | H₂PO₄⁻ |
| B-289 | 2,3-dichlorophenyl | H₂PO₄⁻ |
| B-290 | 2,4-dichlorophenyl | H₂PO₄⁻ |
| B-291 | 2,5-dichlorophenyl | H₂PO₄⁻ |
| B-292 | 2,6-dichlorophenyl | H₂PO₄⁻ |
| B-293 | 3,4-dichlorophenyl | H₂PO₄⁻ |
| B-294 | 3,5-dichlorophenyl | H₂PO₄⁻ |
| B-295 | 2,3-difluorophenyl | H₂PO₄⁻ |
| B-296 | 2,4-difluorophenyl | H₂PO₄⁻ |
| B-297 | 2,5-difluorophenyl | H₂PO₄⁻ |
| B-298 | 2,6-difluorophenyl | H₂PO₄⁻ |
| B-299 | 3,4-difluorophenyl | H₂PO₄⁻ |
| B-300 | 3,5-difluorophenyl | H₂PO₄⁻ |
| B-301 | 2-fluoro-3-chlorophenyl | H₂PO₄⁻ |
| B-302 | 2-fluoro-4-chlorophenyl | H₂PO₄⁻ |
| B-303 | 2-fluoro-5-chlorophenyl | H₂PO₄⁻ |
| B-304 | 2-fluoro-6-chlorophenyl | H₂PO₄⁻ |
| B-305 | 3-fluoro-4-chlorophenyl | H₂PO₄⁻ |
| B-306 | 3-fluoro-5-chlorophenyl | H₂PO₄⁻ |
| B-307 | 2-chloro-3-fluorophenyl | H₂PO₄⁻ |
| B-308 | 2-chloro-4-fluorophenyl | H₂PO₄⁻ |
| B-309 | 2-chloro-5-fluorophenyl | H₂PO₄⁻ |
| B-310 | 3-chloro-4-fluorophenyl | H₂PO₄⁻ |
| B-311 | 2-methyl-3-chlorophenyl | H₂PO₄⁻ |
| B-312 | 2-methyl-4-chlorophenyl | H₂PO₄⁻ |
| B-313 | 2-methyl-5-chlorophenyl | H₂PO₄⁻ |
| B-314 | 2-methyl-6-chlorophenyl | H₂PO₄⁻ |
| B-315 | 3-methyl-4-chlorophenyl | H₂PO₄⁻ |
| B-316 | 3-methyl-5-chlorophenyl | H₂PO₄⁻ |
| B-317 | 2-chloro-3-methylphenyl | H₂PO₄⁻ |
| B-318 | 2-chloro-4-methylphenyl | H₂PO₄⁻ |
| B-319 | 2-chloro-5-methylphenyl | H₂PO₄⁻ |
| B-320 | 3-chloro-4-methylphenyl | H₂PO₄⁻ |
| B-321 | 2-methyl-3-fluorophenyl | H₂PO₄⁻ |
| B-322 | 2-methyl-4-fluorophenyl | H₂PO₄⁻ |
| B-323 | 2-methyl-5-fluorophenyl | H₂PO₄⁻ |
| B-324 | 2-methyl-6-fluorophenyl | H₂PO₄⁻ |
| B-325 | 3-methyl-4-fluorophenyl | H₂PO₄⁻ |
| B-326 | 3-methyl-5-fluorophenyl | H₂PO₄⁻ |
| B-327 | 2-fluoro-3-methylphenyl | H₂PO₄⁻ |
| B-328 | 2-fluoro-4-methylphenyl | H₂PO₄⁻ |
| B-329 | 2-fluoro-5-methylphenyl | H₂PO₄⁻ |
| B-330 | 3-fluoro-4-methylphenyl | H₂PO₄⁻ |
| B-331 | phenyl | CH₃COO⁻ |
| B-332 | 2-chlorophenyl | CH₃COO⁻ |
| B-333 | 3-chlorophenyl | CH₃COO⁻ |
| B-334 | 4-chlorophenyl | CH₃COO⁻ |
| B-335 | 2-fluorophenyl | CH₃COO⁻ |
| B-336 | 3-fluorophenyl | CH₃COO⁻ |
| B-337 | 4-fluorophenyl | CH₃COO⁻ |
| B-338 | 2-methylphenyl | CH₃COO⁻ |
| B-339 | 3-methylphenyl | CH₃COO⁻ |
| B-340 | 4-methylphenyl | CH₃COO⁻ |
| B-341 | 2-methoxyphenyl | CH₃COO⁻ |
| B-342 | 3-methoxyphenyl | CH₃COO⁻ |
| B-343 | 4-methoxyphenyl | CH₃COO⁻ |
| B-344 | 2,3-dichlorophenyl | CH₃COO⁻ |
| B-345 | 2,4-dichlorophenyl | CH₃COO⁻ |
| B-346 | 2,5-dichlorophenyl | CH₃COO⁻ |
| B-347 | 2,6-dichlorophenyl | CH₃COO⁻ |
| B-348 | 3,4-dichlorophenyl | CH₃COO⁻ |
| B-349 | 3,5-dichlorophenyl | CH₃COO⁻ |
| B-350 | 2,3-difluorophenyl | CH₃COO⁻ |
| B-351 | 2,4-difluorophenyl | CH₃COO⁻ |
| B-352 | 2,5-difluorophenyl | CH₃COO⁻ |
| B-353 | 2,6-difluorophenyl | CH₃COO⁻ |
| B-354 | 3,4-difluorophenyl | CH₃COO⁻ |
| B-355 | 3,5-difluorophenyl | CH₃COO⁻ |
| B-356 | 2-fluoro-3-chlorophenyl | CH₃COO⁻ |
| B-357 | 2-fluoro-4-chlorophenyl | CH₃COO⁻ |
| B-358 | 2-fluoro-5-chlorophenyl | CH₃COO⁻ |
| B-359 | 2-fluoro-6-chlorophenyl | CH₃COO⁻ |
| B-360 | 3-fluoro-4-chlorophenyl | CH₃COO⁻ |
| B-361 | 3-fluoro-5-chlorophenyl | CH₃COO⁻ |
| B-362 | 2-chloro-3-fluorophenyl | CH₃COO⁻ |
| B-363 | 2-chloro-4-fluorophenyl | CH₃COO⁻ |
| B-364 | 2-chloro-5-fluorophenyl | CH₃COO⁻ |
| B-365 | 3-chloro-4-fluorophenyl | CH₃COO⁻ |
| B-366 | 2-methyl-3-chlorophenyl | CH₃COO⁻ |
| B-367 | 2-methyl-4-chlorophenyl | CH₃COO⁻ |
| B-368 | 2-methyl-5-chlorophenyl | CH₃COO⁻ |
| B-369 | 2-methyl-6-chlorophenyl | CH₃COO⁻ |
| B-370 | 3-methyl-4-chlorophenyl | CH₃COO⁻ |
| B-371 | 3-methyl-5-chlorophenyl | CH₃COO⁻ |
| B-372 | 2-chloro-3-methylphenyl | CH₃COO⁻ |
| B-373 | 2-chloro-4-methylphenyl | CH₃COO⁻ |
| B-374 | 2-chloro-5-methylphenyl | CH₃COO⁻ |
| B-375 | 3-chloro-4-methylphenyl | CH₃COO⁻ |
| B-376 | 2-methyl-3-fluorophenyl | CH₃COO⁻ |
| B-377 | 2-methyl-4-fluorophenyl | CH₃COO⁻ |
| B-378 | 2-methyl-5-fluorophenyl | CH₃COO⁻ |
| B-379 | 2-methyl-6-fluorophenyl | CH₃COO⁻ |
| B-380 | 3-methyl-4-fluorophenyl | CH₃COO⁻ |
| B-381 | 3-methyl-5-fluorophenyl | CH₃COO⁻ |
| B-382 | 2-fluoro-3-methylphenyl | CH₃COO⁻ |
| B-383 | 2-fluoro-4-methylphenyl | CH₃COO⁻ |
| B-384 | 2-fluoro-5-methylphenyl | CH₃COO⁻ |
| B-385 | 3-fluoro-4-methylphenyl | CH₃COO⁻ |
| B-386 | phenyl | CH₃SO₂O⁻ |
| B-387 | 2-chlorophenyl | CH₃SO₂O⁻ |
| B-388 | 3-chlorophenyl | CH₃SO₂O⁻ |
| B-389 | 4-chlorophenyl | CH₃SO₂O⁻ |
| B-390 | 2-fluorophenyl | CH₃SO₂O⁻ |
| B-391 | 3-fluorophenyl | CH₃SO₂O⁻ |
| B-392 | 4-fluorophenyl | CH₃SO₂O⁻ |
| B-393 | 2-methylphenyl | CH₃SO₂O⁻ |
| B-394 | 3-methylphenyl | CH₃SO₂O⁻ |
| B-395 | 4-methylphenyl | CH₃SO₂O⁻ |
| B-396 | 2-methoxyphenyl | CH₃SO₂O⁻ |
| B-397 | 3-methoxyphenyl | CH₃SO₂O⁻ |
| B-398 | 4-methoxyphenyl | CH₃SO₂O⁻ |
| B-399 | 2,3-dichlorophenyl | CH₃SO₂O⁻ |
| B-400 | 2,4-dichlorophenyl | CH₃SO₂O⁻ |
| B-401 | 2,5-dichlorophenyl | CH₃SO₂O⁻ |
| B-402 | 2,6-dichlorophenyl | CH₃SO₂O⁻ |
| B-403 | 3,4-dichlorophenyl | CH₃SO₂O⁻ |
| B-404 | 3,5-dichlorophenyl | CH₃SO₂O⁻ |
| B-405 | 2,3-difluorophenyl | CH₃SO₂O⁻ |
| B-406 | 2,4-difluorophenyl | CH₃SO₂O⁻ |
| B-407 | 2,5-difluorophenyl | CH₃SO₂O⁻ |
| B-408 | 2,6-difluorophenyl | CH₃SO₂O⁻ |
| B-409 | 3,4-difluorophenyl | CH₃SO₂O⁻ |
| B-410 | 3,5-difluorophenyl | CH₃SO₂O⁻ |
| B-411 | 2-fluoro-3-chlorophenyl | CH₃SO₂O⁻ |
| B-412 | 2-fluoro-4-chlorophenyl | CH₃SO₂O⁻ |
| B-413 | 2-fluoro-5-chlorophenyl | CH₃SO₂O⁻ |
| B-414 | 2-fluoro-6-chlorophenyl | CH₃SO₂O⁻ |
| B-415 | 3-fluoro-4-chlorophenyl | CH₃SO₂O⁻ |
| B-416 | 3-fluoro-5-chlorophenyl | CH₃SO₂O⁻ |
| B-417 | 2-chloro-3-fluorophenyl | CH₃SO₂O⁻ |
| B-418 | 2-chloro-4-fluorophenyl | CH₃SO₂O⁻ |
| B-419 | 2-chloro-5-fluorophenyl | CH₃SO₂O⁻ |
| B-420 | 3-chloro-4-fluorophenyl | CH₃SO₂O⁻ |
| B-421 | 2-methyl-3-chlorophenyl | CH₃SO₂O⁻ |
| B-422 | 2-methyl-4-chlorophenyl | CH₃SO₂O⁻ |
| B-423 | 2-methyl-5-chlorophenyl | CH₃SO₂O⁻ |
| B-424 | 2-methyl-6-chlorophenyl | CH₃SO₂O⁻ |
| B-425 | 3-methyl-4-chlorophenyl | CH₃SO₂O⁻ |
| B-426 | 3-methyl-5-chlorophenyl | CH₃SO₂O⁻ |
| B-427 | 2-chloro-3-methylphenyl | CH₃SO₂O⁻ |
| B-428 | 2-chloro-4-methylphenyl | CH₃SO₂O⁻ |
| B-429 | 2-chloro-5-methylphenyl | CH₃SO₂O⁻ |
| B-430 | 3-chloro-4-methylphenyl | CH₃SO₂O⁻ |
| B-431 | 2-methyl-3-fluorophenyl | CH₃SO₂O⁻ |
| B-432 | 2-methyl-4-fluorophenyl | CH₃SO₂O⁻ |
| B-433 | 2-methyl-5-fluorophenyl | CH₃SO₂O⁻ |
| B-434 | 2-methyl-6-fluorophenyl | CH₃SO₂O⁻ |
| B-435 | 3-methyl-4-fluorophenyl | CH₃SO₂O⁻ |
| B-436 | 3-methyl-5-fluorophenyl | CH₃SO₂O⁻ |
| B-437 | 2-fluoro-3-methylphenyl | CH₃SO₂O⁻ |
| B-438 | 2-fluoro-4-methylphenyl | CH₃SO₂O⁻ |
| B-439 | 2-fluoro-5-methylphenyl | CH₃SO₂O⁻ |
| B-440 | 3-fluoro-4-methylphenyl | CH₃SO₂O⁻ |

According to the inventive process, the hydrazine derivative (II) and/or a salt (IIa) thereof, as defined above, is reacted with a thiocyanate Mn⁴(SCN)ₙ (IV) and an orthoformic ester HC(OR⁵)(OR⁶)(OR⁷) (V),

According to one inventive embodiment of the process, variable Mⁿ⁺ in the thiocyanate Mⁿ⁺(SCN)ₙ (IV) stands for an alkali metal cation (thus, n=1), an alkaline earth metal cation (for example Ca or Mg; thus, n=2), Ag⁺ (n=1), Cu²⁺ (n=2), Co²⁺ (n=2), Cd²⁺ (n=2) or Fe³⁺ (n=3). In one embodiment, Mⁿ⁺ stands for an alkali metal cation (n=1). An “alkali metal cation” means in particular Na⁺ or K. Thus, preferred thiocyanates are NaSCN and KSCN.

n is 1, 2 or 3, depending on the meaning of M.

According to another inventive embodiment of the process, variable Mⁿ⁺ in the thiocyanate Mⁿ⁺(SCN)ₙ (IV) stands for ammonium or alkylammonium [NR¹R²R³R⁴]⁺, wherein R¹, R², R³ and R⁴ independently from each other in [NR¹R²R³R⁴]⁺ stand for hydrogen or (C₁-C₁₀) alkyl. According to one specific embodiment of the invention, the thiocyanate used is NH₄SCN. According to another specific embodiment of the invention, R¹, R², R³, R⁴ independently stand for (C₁-C₁₀)alkyl, in particular (C₁-C₆)alkyl, more particularly (C₁-C₄)alkyl. According to one specific aspect, R¹, R², R³, R⁴ have the same meaning and stand for (C₁-C₁₀)alkyl, in particular (C₁-C₆)alkyl, more particularly (C₁-C₄)alkyl. Specific examples for such thiocyanates (IV) are N(CH₃)₄SCN and N(C₂H₅)₄SCN.

Thiocyanates Mⁿ⁺(SCN)ₙ (IV) are well known in the art and are commercially available.

The use of an orthoformic ester HC(OR⁵)(OR⁶)(OR⁷) is characteristic for the inventive process. R⁵, R⁶, R⁷ are independently from each other selected from C₁-C₈-alkyl, C₂-C₈-alkenyl and C₂-C₈-alkinyl, preferably selected from C₁-C₈-alkyl. According to one embodiment, R⁵, R⁶ and R⁷ are the same (in the following named HC(OR⁵)₃) and selected from C₁-C₈-alkyl, C₂-C₃-alkenyl and C₂-C₈-alkinyl, preferably selected from C₁-C₈-alkyl, more particularly selected from C₁-C₄-alkyl. Specific meanings for R⁵, R⁶, and R⁷, which can be the same or different from each other, are methyl and ethyl. It may be preferred according to the present invention to use orthoformic triethyl ester or orthoformic trimethyl ester, in particular since they are easily available and cheap.

The preparation of orthoformic esters HC(OR⁵)(OR⁶)(OR⁷) and, particularly HC(OR⁵)₃ is well-known to the skilled person, some of them are commercially available.

According to the inventive process the reactants can be added in any order to the reaction flask or vessel. According to one embodiment, the hydrazine (II) and/or salt (IIa) is added first. Then the thiocyanate and the orthoformic ester can be added together or subsequently. If added subsequently, either the thiocyanate or the orthoformic ester can be added first. According to another embodiment of the invention, the thiocyanate is first added to the reaction mixture comprising the hydrazine derivative, then the orthoformic ester is added. According to still another embodiment, the thiocyanate and the orthoformic ester are added to the hydrazine derivative at the same time, either together or separately, preferably separately.

According to the inventive process for preparing compounds of formula I from hydrazine derivatives of formula (II) and/or (IIa), the reaction is carried out without the addition of formic acid as reactant.

As diluting agent for the inventive process any organic solvents, in particular polar or non-polar organic solvents. Preferably, a polar organic solvent is used, in particular a polar protic solvent. It may be preferred if organic carbonic acids are used such as for example acetic acid. In one specific embodiment, glacial acetic acid is used.

In general, the inventive process can be carried out within a certain temperature range. In particular it may be preferred if temperatures of 20° C. to 80° C., in particular 20° C. to 60° C., more particularly 20° C. to 55° C. are used. Furthermore, it may be preferred if temperatures of 25° C. to 75° C., in particular 25° C. to 65° C., more particularly 30° C. to 55° C. are used.

It may be preferred in the inventive process, if 1 to 5 moles of the orthoformic ester HC(OR⁵)(OR⁶)(OR⁷) are used compared to the hydrazine starting compound (II) and/or (IIa), preferably, 1 to 2 moles of the orthoformic ester are used.

Furthermore, it may be preferred in the inventive process, if 1 to 5 moles of the thiocyanate (IV) are used for one mol hydrazine starting compound (II) and/or (IIa), preferably, 1 to 2 moles of the thiocyanate (IV) are used.

One advantage of the inventive process is that the cyclization reaction to the thiotriazolo compound can be carried out in a one-pot reaction. Furthermore, if desired, the reaction can be carried out at temperatures that are comparably low (40° C. to 60° C.) and the reaction times are comparably short. The process is thus very economic.

In order to obtain compounds of formula (I) that contain a derivatized sulfur group, the compounds of formula (I) can be further reacted according to processes known in the art. Such compounds are also useful for combating phytopathogenic fungi.

For example, by further reaction of compounds (I) with R⁸—X, where R⁸ is as defined below and X is a leaving group such as, for example, halogen, such as Cl, Br or I, or trifluoro-C₁-C₆-alkylsulfonate, it is possible to prepare various compounds of the formula (I) carrying a S—R′ group instead of “S—H”. To prepare compounds containing a group SR⁸ where R⁸ is C₁-C₈-alkyl, preferably is C₁-C₄-alkyl, in particular methyl or ethyl, a compound (I) is reacted with the corresponding alkyl halide (see also WO 96/38440).

Further, the following S-residues can be formed from the respective SH-derivative of formula (I):

S—R⁸, where
R⁸ is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R⁹, C(═S)R⁹, SO₂R¹⁹ or CN; where
R⁹ is C₁-C₈-alkyl, C₁-C₈-haloalkyl, C₁-C₈-alkoxy, C₁-C₈-haloalkoxy or NA³A⁴; and
R¹⁰ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
A³, A⁴ independently of one another are hydrogen, C₁-C₈-alkyl, C₁-C₈-haloalkyl, C₂-C₃-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl

S-DII, wherein DII is

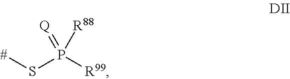

where # is the point of attachment to the triazolyl ring and Q, R⁸⁸ and R⁹⁹ are as defined below:
Q is O or S;
R⁸⁸, R⁹⁹ independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR¹¹¹R²²², where R¹¹¹ is H or C₁-C₈-alkyl and R²²² is C₁-C₈-alkyl, phenyl-C₁-C₄-alkyl or phenyl or R¹¹¹ and R²²² together are an alkylene chain having four or five carbon atoms or form a radical of the formula-CH₂—CH₂—O—CH₂—CH₂— or —CH₂—CH₂—NR³³³—CH₂—CH₂— in which R³³³ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;

SM¹, where M¹ is as defined below:
M¹ is an alkali metal cation, an equivalent of an alkaline earth metal cation, an equivalent of a copper, zinc, iron or nickel cation or an ammonium cation of the formula (E)

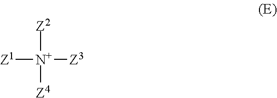

in which
Z¹ and Z² independently are hydrogen or C₁-C₈-alkyl;
Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;

Compounds of the formula I which contain a group S—C(═O)NA³A⁴ can be synthesized analogously to the process described in WO 99/21853.

Compounds of the formula I which contain a group DII can be synthesized analogously to the process described in WO 99/05149.

Compounds of the formula I which contain a group S—SO₂R¹⁰ can be synthesized analogously to the process described in WO 97/44332.

Compounds of the formula I which contain a group S—CN can be synthesized analogously to the process described in WO 99/44331.

Compounds of the formula I which contain a group DI can be synthesized analogously to the process described in WO 97/43269.

Compounds of the formula I which contain a group S—C(═O)R⁹ where R⁹═C₁-C₈-alkyl, C₁-C₈-haloalkyl, C₁-C₈-alkoxy or C₁-C₈-haloalkoxy can be synthesized analogously to the process described in WO 97/42178.

Compounds of the formula I which contain a group SM¹ can be synthesized analogously to the process described in WO 97/41107.

According to one aspect of the present invention, one of the steps for derivatizing the sulfur in the triazole ring as detailed above is carried out following the process of the present invention. According to one specific aspect, following the synthesis of compounds (I)-(1) according to the process of the present invention, one of the steps for derivatizing the sulfur in the triazole ring is carried out. This represents a very useful approach for the synthesis of further fungicidal compounds, in particular where SH is derivatized into SR⁸, R⁸ being C₁-C₈-alkyl, in particular C₁-C₄-alkyl, C₂-C₈-alkenyl or CN.

### EXAMPLES

The following examples further illustrate the present invention and do not restrict the invention in any manner.

### Example 1

### Synthesis of 2-[rel-(2S,3R)-3-(2-Chlorophenyl)-2-(2,4-difluorophenyl)-oxiranylmethyl]-2,4-dihydro-[1,2,4]triazol-3-thione

To a solution of methanesulfonic acid-3-(2-chlorophenyl)-2-(2,4-difluorophenyl)-oxiranylmethyl ester (20.0 g, 53.4 mmol) in isopropanol (40 ml) hydrazinhydrate (25.89 ml, 533.6 mmol) was added, and the reaction mixture was stirred for 1.5 hours under reflux. After the reaction mixture had cooled down to room temperature, water and MTBE were added, the organic layer was separated and washed with water. The organic phase was dried, concentrated under reduced pressure and the residue was taken up in acetic acid (200 ml). Sodium thiocyanate (4.54 g, 56.0 mmol) was added and then, the reaction mixture was warmed up to 40° C. and triethylorthoformiate (8.30 g, 56.0 mmol) was added dropwise. After completion of the reaction the mixture was poured onto ice water (1000 ml). The resulting residue was filtered off. The resulting solid was taken up with ethyl acetate and dried with sodium sulfate. After removal of the solvent under reduced pressure the title compound was obtained in form of a beige solid in a purity of 80% (18.0 g, 71%).

### Example 2

### Synthesis of 2-[rel-(2S,3R)-3-(2-Chloro-phenyl)-2-(2,4-difluoro-phenyl)-oxiranylmethyl]-2,4-dihydro-[1,2,4]triazole-3-thione starting from [3-(2-Chloro-phenyl)-2-(2,4-difluoro-phenyl)-oxiranylmethyl]-hydrazinium phosphate

### 2.1 Synthesis of [3-(2-Chloro-phenyl)-2-(2,4-difluorophenyl)-oxiranylmethyl]-hydrazinium phosphate

To a solution of hydrazine hydrate (25.04 g, 0.50 mol) in acetonitrile (5 ml) was added a solution of methanesulfonic acid 3-(2-chloro-phenyl)-2-(2,4-difluorophenyl)-oxiranylmethyl ester (37.5 g, 0.10 mol) in toluene (150 ml). The mixture was heated to 85° C. while being intensively stirred for 30 h, after which time the starting material was completely consumed. The mixture was then cooled to ambient temperature before being washed with water (3×150 ml). The organic phase was separated, dried over Na₂SO₄ and used in the next step without purification. The solution obtained above was diluted with toluene (200 ml) and treated dropwise with H₃PO₄ (17.29 g, 0.15 mol) at room temperature. The mixture was stirred overnight, during which time a precipitate formed. The solid was filtered off, washed with toluene then heptane and dried, affording the target product as brownish solid (31.0 g, 76%). ¹H-NMR (360 MHz, DMSO-d₆): δ7.602-7.11 (m, 7H), 5.40 (br s, 4H), 4.20 (s, 1H), 3.20 (d, 1H), 2.59 (d, 1H).

### 2.2 Synthesis of 2-[rel-(2S,3R)-3-(2-Chloro-phenyl)-2-(2,4-difluoro-phenyl)-oxiranylmethyl]-2,4-dihydro-[1,2,4]triazole-3-thione

[3-(2-Chloro-phenyl)-2-(2,4-difluoro-phenyl)-oxiranylmethyl]-hydrazinium phosphate (10.2 g, 2.5 mmol) and NaSCN (0.22 g, 2.75 mmol) were dissolved in glacial acetic acid (15 ml) and heated to 60° C. At this temperature, HC(OEt)₃ (0.78 g, 5.25 mmol) was added dropwise while stirring. After completion of the reaction, the mixture was poured in water and extracted with ethyl acetate (3×50 ml). The combined organic phases were washed with brine and dried. The solid obtained after removal of the sol-vent in vacuo proved to be the target product in 80% purity (1.00 g, 84%).

### Example 3

### Synthesis of 2-[rel-(2S,3R)-2-(3,4-Difluoro-phenyl)-3-o-tolyl-oxiranylmethyl]-2,4-dihydro-[1,2,4]triazole-3-thione

To a solution of Methanesulfonic acid 2-(3,4-difluoro-phenyl)-3-o-tolyl-oxiranylmethyl ester (7.50 g, 19.1 mmol) in isopropanol (20 ml) was added hydrazine hydrate (11.55 ml, 190.5 mmol). The mixture was heated to reflux and stirred for 1.5 hours. After this time, the reaction mixture was partitioned between MTBE and water and the organic phase washed with water and dried (Na₂SO₄). The residue obtained after removal of the solvent was dissolved in glacial acetic acid (70 ml) and treated with NaSCN (1.70 g, 21.0 mmol) and triethylorthoformat HC(OEt)₃ (3,105 g, 20.95 mmol) at 50° C. Subsequently, the mixture was cooled to room temperature and stirring was continued for 2 h, after which time the starting material was completely consumed. The mixture was then poured in ice water and the precipitate was filtered off. The solid thus obtained was purified with column chromatography (silica, ethyl acetate/hexanes). Collection of the appropriate fractions afforded the target compounds as yellowish solid (1.80 g, 25%). ¹H-NMR (400 MHz, CDCl₃): (11.75 (br s, 1H), 7.70 (s, 1H), 7.71-7-06 (m, 7H), 5.03 (d, 1H), 3.99 (s, 1H), 3.71 (d, 1H), 2.38 (s, 3H).

1-3. (canceled)

4. A process for preparing the compound of formula (I) 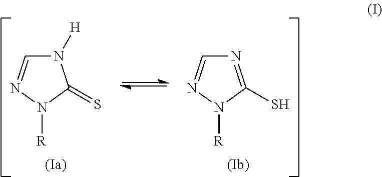
comprising reacting a compound of formula (II) 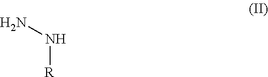
and/or a salt (IIa) thereof, 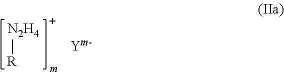
with Mⁿ⁺(SCN)ₙ (IV) and an orthoformic ester HC(OR⁵)(OR⁶)(OR⁷) (V), wherein:
R is
(1): 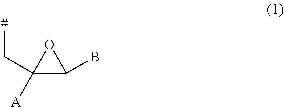
wherein # shall mean the point of attachment to the triazolo group or the hydrazine unit, respectively, wherein:
A or B is a three-, four-, five-, six-, seven-, eight-, nine- or ten-membered saturated or partially unsaturated heterocycle or five-, six-, seven-, eight-, nine- or ten-membered aromatic heterocycle, where the heterocycle contains in each case one, two, three or four heteroatoms from the group consisting of O, N and S; is naphthyl or phenyl;
and the respective other variable B or A has one of the meanings mentioned above for A or B or is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl or benzodioxolyl;
where A and B independently of one another are unsubstituted or substituted by one, two, three or four independently selected substituents L; wherein
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl-C₁-C₈-alkyl, phenyl, phenyloxy or a five- or six-membered saturated, partially unsaturated or aromatic heterocycle which contains one, two, three or four heteroatoms selected from the group consisting of O, N and S; wherein:
n is 0, 1 or 2;
A¹ is hydrogen, hydroxyl, C₁-C₈-alkyl, C₁-C₈-haloalkyl, amino, C₁-C₈-alkylamino or di-C₁-C₈-alkylamino,
A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halo-cycloalkoxy;
A³, A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
where the aliphatic, alicyclic or aromatic groups of the radical definitions of L for their part may carry one, two, three or four identical or different groups R^{L}:
R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, or di-C₁-C₈-alkylamino;
(2): 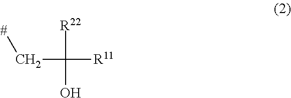
wherein:
R¹¹, R²² independently of one another are C₁-C₆-alkyl, C₁-C₆-haloalkyl, C₃-C₆-cycloalkyl, C₃-C₆-halocycloalkyl or phenyl, wherein the alkyl, cycloalkyl and phenyl moieties may be unsubstituted or substituted by one, two or three substituents L as defined above for compounds wherein R is a group (1); or
R¹¹ and R²², together with the carbon atom to which they are attached, form a five- or six-membered saturated or partially unsaturated ring, that can be unsubstituted or substituted by one, two, three, four or five substituents L′, wherein L′ stands for L as defined above or stands for a group 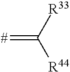
wherein R³³ and R⁴⁴ independently are selected from the group consisting of hydrogen and L as defined above; or
(3): 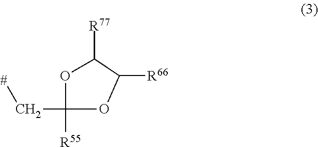
wherein:
R⁵⁵ is phenyl-C₁-C₈-alkyl, phenyl or a five- or six-membered saturated, partially unsaturated or aromatic heterocycle which contains one, two, three or four heteroatoms selected from the group consisting of O, N and S; where the aliphatic, aromatic, or heterocyclic groups for their part may carry one, two, three or four identical or different groups selected from the group consisting of halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈alkylcarbonyl, C₁-C₈-alkylcarbonyloxy, C₁-C₈-alkoxycarbonyl, amino, C₁-C₈-alkylamino, di-C₁-C₈-alkylamino, phenyl, halophenyl, phenyloxy, and halophenyloxy;
R⁶⁶, R⁷⁷ are independently of one another hydrogen, C₁-C₆-alkyl, C₁-C₆-haloalkyl, C₃-C₆-cycloalkyl, C₃-C₆-halocycloalkyl or phenyl, wherein the alkyl, cycloalkyl or phenyl moieties may be unsubstituted or substituted by one, two or three substituents selected from halogen, cyano, nitro, C₁-C₈-alkyl, C₁-C₈-haloalkyl, C₁-C₈-alkoxy, or C₁-C₈-haloalkoxy;
n is 1, 2 or 3, depending on the meaning of M;
Mⁿ⁺ alkali metal cation (n=1), alkaline earth metal cation (n=2), A_{g}+(n=1), Cu²⁺ (n=2), Co²⁺ (n=2), Cd²⁺ (n=2), Fe³⁺ (n=3); or [NR¹R²R³R⁴]⁺, wherein R¹, R², R³ and R⁴ independently from each other are selected from the group consisting of hydrogen and (C₁-C₁₀)-alkyl;
R⁵, R⁶, R⁷ independently from each other are selected from the group consisting of C₁-C₈-alkyl, C₂-C₈-alkenyl and C₂-C₈-alkinyl;
Y^{m-} is a counter anion of an organic acid or an inorganic acid and
m is 1, 2 or 3.

5. The process according to claim 4, wherein R has the following meaning (1): 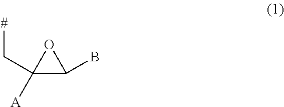
wherein # shall mean the point of attachment to the triazolo group or the hydrazine unit, respectively, and:
A or B is naphthyl, phenyl, a three-, four-, five-, six-, seven-, eight-, nine- or ten-membered saturated or partially unsaturated heterocycle or a five-, six-, seven-, eight-, nine- or ten-membered aromatic heterocycle, where the heterocycle contains in each case one, two, three or four heteroatoms selected from the group consisting of O, N and S;
and the respective other variable B or A has one of the meanings mentioned above for A or B or is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl or benzodioxolyl;
where A and B independently of one another are unsubstituted or substituted by one, two, three or four independently selected substituents L; wherein
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl-C₁-C₈-alkyl, phenyl, phenyloxy or a five- or six-membered saturated, partially unsaturated or aromatic heterocycle which contains one, two, three or four heteroatoms selected from the group consisting of O, N and S; wherein:
n is 0, 1 or 2;
A¹ is hydrogen, hydroxyl, C₁-C₈-alkyl, C₁-C₈-haloalkyl, amino, C₁-C₈-alkylamino or di-C₁-C₈-alkylamino,
A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
A³, A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
where the aliphatic, alicyclic, or aromatic groups of the radical definitions of L for their part may carry one, two, three or four identical or different groups R^{L}:
R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, or di-C₁-C₈-alkylamino.

6. A compound of formula (IIa), 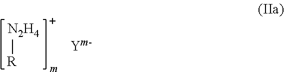
wherein:
R is
(1): 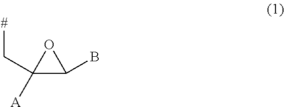
wherein # shall mean the point of attachment to the triazolo group or the hydrazine unit, respectively, wherein:
A or B is a three-, four-, five-, six-, seven-, eight-, nine- or ten-membered saturated or partially unsaturated heterocycle or five-, six-, seven-, eight-, nine- or ten-membered aromatic heterocycle, where the heterocycle contains in each case one, two, three or four heteroatoms from the group consisting of O, N and S; is naphthyl or phenyl;
and the respective other variable B or A has one of the meanings mentioned above for A or B or is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl or benzodioxolyl;
where A and B independently of one another are unsubstituted or substituted by one, two, three or four independently selected substituents L; wherein
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl-C₁-C₈-alkyl, phenyl, phenyloxy or a five- or six-membered saturated, partially unsaturated or aromatic heterocycle which contains one, two, three or four heteroatoms selected from the group consisting of O, N and S; wherein:
n is 0, 1 or 2;
A¹ is hydrogen, hydroxyl, C₁-C₈-alkyl, C₁-C₈-haloalkyl, amino, C₁-C₈-alkylamino or di-C₁-C₈-alkylamino,
A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
A³, and A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
where the aliphatic, alicyclic or aromatic groups of the radical definitions of L for their part may carry one, two, three or four identical or different groups R^{L}:
R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, or di-C₁-C₈-alkylamino;
(2): 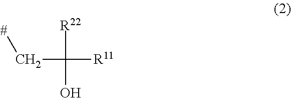
wherein:
R¹¹, and R²² independently of one another are C₁-C₆-alkyl, C₁-C₆-haloalkyl, C₃-C₆-cycloalkyl, C₃-C₆-halocycloalkyl or phenyl, wherein the alkyl, cycloalkyl and phenyl moieties may be unsubstituted or substituted by one, two or three substituents L as defined above for compounds wherein R is a group (1); or
R¹¹ and R²², together with the carbon atom to which they are attached, form a five- or six-membered saturated or partially unsaturated ring, that can be unsubstituted or substituted by one, two, three, four or five substituents L′, wherein L′ stands for L as defined above or stands for a group 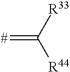
wherein R³³ and R⁴⁴ independently are selected from the group consisting of hydrogen and L as defined above; or
(3): 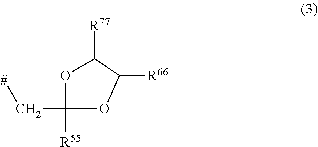
wherein:
R⁵⁵ is phenyl-C₁-C₈-alkyl, phenyl or a five- or six-membered saturated, partially unsaturated or aromatic heterocycle which contains one, two, three or four heteroatoms selected from the group consisting of O, N and S; where the aliphatic, aromatic, or heterocyclic groups for their part may carry one, two, three or four identical or different groups selected from the group consisting of halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino, phenyl, halophenyl, phenyloxy, and halophenyloxy;
R⁶⁶, R⁷⁷ are independently of one another hydrogen, C₁-C₆-alkyl, C₁-C₆-haloalkyl, C₃-C₆-cycloalkyl, C₃-C₆-halocycloalkyl or phenyl, wherein the alkyl, cycloalkyl or phenyl moieties may be unsubstituted or substituted by one, two or three substituents selected from halogen, cyano, nitro, C₁-C₈-alkyl, C₁-C₈-haloalkyl, C₁-C₈-alkoxy, or C₁-C₈-haloalkoxy;
Y^{m-} is a counter anion of an organic acid or an inorganic acid and
m is 1, 2 or 3.
